(19) United States
(12) Patent Application Publication
(10) Pub. No.: US 2024/0382518 A1
(43) Pub. Date: Nov. 21, 2024

(54) PHARMACEUTICAL COMPOSITION AND HEALTH FUNCTIONAL FOOD FOR PREVENTION OR TREATMENT OF OSTEOARTHRITIS

(71) Applicant: SEOUL NATIONAL UNIVERSITY R&DB FOUNDATION, Seoul (KR)

(72) Inventors: JIN HONG KIM, Seoul (KR); DONG HYUN KANG, Seoul (KR); JEE YEON LEE, Seoul (KR)

(21) Appl. No.: 18/692,447

(22) PCT Filed: Sep. 19, 2022
(86) PCT No.: PCT/KR2022/013994

§ 371 Date: Mar. 15, 2024

(30) Foreign Application Priority Data
Sep. 17, 2021 (KR) 10-2021-0125067
Sep. 19, 2022 (KR) 10-2022-0117850

(51) Int. Cl. A61K 33/04; A61K 31/198; A61K 31/375; A61K 38/06; A61P 19/02

(52) U.S. Cl. CPC A61K 33/04; A61K 31/198; A61K 31/375; A61K 38/063; A61P 19/02

(57) ABSTRACT

A composition according to an embodiment includes a selenium-containing material and an antioxidant. The composition may inhibit degeneration of cartilage by a mechanism such as reducing oxidative stress in cells constituting the cartilage. Accordingly, it is possible to inhibit an occurrence of osteoarthritis, delay its progression, or enable treatment of osteoarthritis.

### CROSS REFERENCE TO RELATED APPLICATIONS AND CLAIM OF PRIORITY

This application claims benefit under 35 U.S.C. 119, 120, 121, or 365 (c), and is a National Stage entry from International Application No. PCT/KR2022/013994, filed Sep. 19, 2022, which claims priority to the benefit of Korean patent application Nos. 10-2021-0125067 filed on Sep. 17, 2021, and 10-2022-0117850 filed on

Sep. 19, 2022, in the Korean intellectual property office, the entire contents of which are incorporated herein by reference.

### BACKGROUND

### 1. Technical Field

The present invention relates to a pharmaceutical composition for preventing or treating osteoarthritis, and a health functional food for preventing or improving osteoarthritis.

### 2. Background Art

Osteoarthritis is a representative degenerative disease of which incidence is rapidly increasing with aging of the population. It is caused by the gradual and irreversible degeneration of cartilage tissues constituting a joint, hence resulting in abnormalities in exercise ability. Further, in severe cases, an inflammatory reaction in the cartilage may cause severe pain. Aging, physical cartilage tissue damage, cartilage matrix homeostasis imbalance, reduction of regulatory factors involved in cartilage regeneration, etc. have been suggested as pathological causes of cartilage tissue degeneration in patients with osteoarthritis. However, any essential treatment measure is still not developed. Currently, the treatment of osteoarthritis focuses on surgical operation or drug treatment. In the case of drug treatment, anti-inflammatory drugs or chondroprotective agents such as hyaluronic acid or glucosamine are used to only relieve pain, however, for the purpose of regenerating chondrocytes or modulating degeneration, any therapeutics with verified effects have yet to be developed.

Osteoarthritis is caused by various causes including systemic factors due to aging or local factors such as mechanical/physical stress due to excessive body weight and joint instability. It is known that such aging and abnormal mechanical stress accelerate the accumulation of oxidative stress in chondrocytes, which in turn can trigger sub-step mechanisms such as cellular senescence, dedifferentiation and apoptosis.

Selenium corresponds to a trace element that plays a physiological role through a selenoprotein containing selenocysteine at an active site. Although it has been found that some selenoproteins have antioxidant functions, it is unclear whether selenium, a component of selenoproteins, can have therapeutic or alleviating effects on osteoarthritis, and a mechanism of selenoprotein involved in the pathological mechanism of osteoarthritis is also not clearly known. Accordingly, it is urgent to develop a new mechanism of osteoarthritis treatment measure using materials related to selenium metabolism.

### SUMMARY

An object of the present invention is to provide a pharmaceutical composition for preventing or treating osteoarthritis

Another object of the present invention is to provide a health functional food for preventing or improving osteoarthritis.
1. A pharmaceutical composition for preventing or treating osteoarthritis, including a selenium-containing material and an antioxidant.
2. The pharmaceutical composition according to the above 1, wherein the selenium-containing material is at least one material selected from the group consisting of selenite, selenate or selenomethionine (SeMet), or a salt thereof.
3. The pharmaceutical composition according to the above 1, wherein the antioxidant is at least one selected from the group consisting of N-acetylcysteine, ascorbic acid or glutathione.
4. The pharmaceutical composition according to the above 1, wherein the selenium-containing material is included in an amount of 0.1 nM to 10 μM, or 1 ppm to 2,000 ppm in the total composition.
5. The pharmaceutical composition according to the above 1, wherein the antioxidant is included in an amount of 1 μM to 10 mM, or 1,000 ppm to 500,000 ppm in the total composition.
6. A health functional food for preventing or improving osteoarthritis, including a selenium-containing material and an antioxidant.
7. The health functional food according to the above 6, wherein the selenium-containing material is at least one material selected from the group consisting of selenite, selenate, or selenomethionine (SeMet), or a salt thereof.
8. The health functional food according to the above 6, wherein the antioxidant is at least one selected from the group consisting of N-acetylcysteine, ascorbic acid or glutathione.
9. The health functional food according to the above 6, wherein the selenium-containing material is included in an amount of 0.1 nM to 10 μM, or 1 ppm to 2,000 ppm in the total composition.
10. The health functional food according to the above 6, wherein the antioxidant is included in an amount of 1 μM to 10 mM, or 1,000 ppm to 500,000 ppm in the total composition.

The pharmaceutical composition or health functional food including a selenium-containing material and an antioxidant according to the present invention can inhibit degeneration of cartilage by a mechanism including reducing oxidative stress in cells constituting the cartilage. Accordingly, it is possible to inhibit an occurrence of osteoarthritis, delay its progression, or enable treatment of osteoarthritis.

### BRIEF DESCRIPTION OF THE DRAWINGS

FIG. 1 is a schematic view of the degeneration regulatory mechanism of cartilage tissue by selenophosphate synthetase 1 (SEPHS1) among selenium metabolic factors.

FIGS. 2A to 2F illustrate results of confirming that the expression of SEPHS1 is reduced in the cartilage of a mouse or human with osteoarthritis.

FIGS. 3A to 3U illustrate results of confirming that the decrease in expression of SEPHS1 induces cellular senescence by oxidative stress in chondrocytes.

FIGS. 4A to 4G illustrate results of analysis for the natural aging-associated osteoarthritis phenotype in SEPHS1 knockout mice.

FIGS. 5A to 5I illustrate results of analysis for the post-traumatic osteoarthritis phenotype in SEPHS1 knockout mice.

FIGS. 6A to 6G illustrate results of analysis for the osteoarthritis phenotype induced in SEPHS1 knockout mice with or without dietary antioxidant treatment.

FIGS. 7A to 7M illustrate results similar to those in the case where osteoarthritis is exacerbated in selenophosphate synthetase 1 (SEPHS1) knockout mice treated with selenium-deficient diet, confirming that dietary selenium deficiency and SEPHS1 knockout show complementary effects of exacerbating osteoarthritis.

FIGS. 8A to 8D illustrate results of analysis for expression level of matrix metalloproteinase 13 (MMP13) or ADAMTS5 (A disintegrin and metalloproteinase with thrombospondin motifs 5) by individual/combined administration of selenium-containing materials or/and antioxidants.

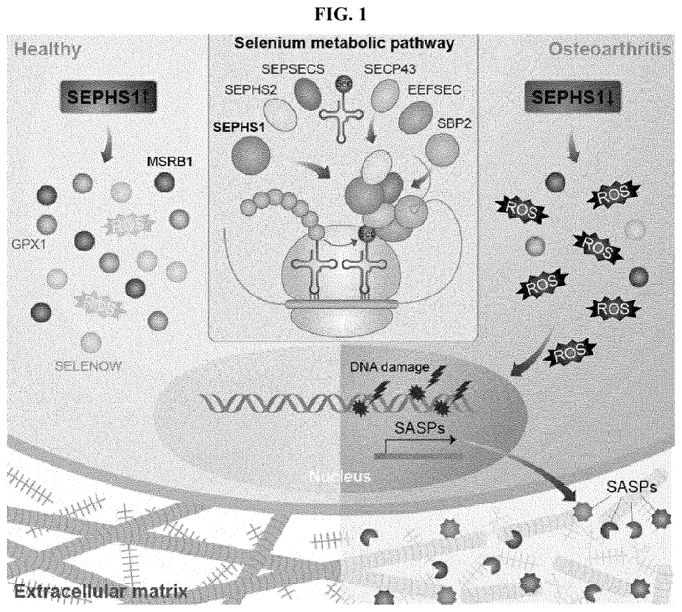

### DETAILED DESCRIPTION

Hereinafter, the present invention will be described in detail. Unless otherwise specifically defined, all terms in the present specification would have the same meanings as general meanings of the corresponding terms understood by persons having common knowledge to which the present invention pertains (“those skilled in the art”), and if the general meanings conflict with the meanings of the terms used herein, the meanings used in the present specification take precedence.

The present invention relates to a pharmaceutical composition for preventing or treating osteoarthritis, which includes a selenium-containing material and an antioxidant.

The pharmaceutical composition of the present invention may exhibit excellent preventive or therapeutic effects on osteoarthritis due to the combination of a selenium-containing material and an antioxidant. The selenium-containing material may accelerate the synthesis of selenoproteins, for example, one or more proteins selected from the group including methionine sulfoxide reductase B1 (MSRB1), glutathione peroxidase 1 (GPX1), selenoprotein T (SELENOT) or selenoprotein W (SELENOW) so as to prevent aging of chondrocytes. More specifically, treatment with a material containing selenium may play a role in protecting oxidative stress-induced DNA damage, such as an increase in the expression of the selenoprotein gene associated with cellular stress, increase in the synthesis of protein, or increase in the activity of protein, etc inside cartilage. It may suppress the secretion of senescence-associated secretory phenotypes (SASPs) such as matrix metalloproteinase (MMP) and ADAMTS (A disintegrin and metalloproteinase with thrombospondin motifs), and inhibit degradation of the extracellular matrix of chondrocytes, thereby preventing degeneration of the cartilage matrix while inhibiting an occurrence of osteoarthritis or its progression. Further, the antioxidant may maximize the effect of suppressing oxidative stress caused by the selenium-containing material, thereby further increasing the effect of inhibiting the occurrence of osteoarthritis or its progression. However, it is not limited thereto.

In the present invention, the selenium-containing material may be included regardless of its type or chemical formula. For example, the material may be selenite, selenate or selenomethionine (SeMet), or a pharmaceutically acceptable salt thereof, but it is not limited thereto.

In the present invention, the antioxidant may be included without limitation to its type or chemical formula as long as it inhibits oxidative stress. For example, it may inhibit oxidative stress by suppressing reactive oxygen species (ROS) such as superoxide, hydrogen peroxide, and hydroxyl radical, and more specifically, it may be N-acetylcysteine, ascorbic acid or glutathione, but it is not limited thereto.

In the present invention, the concentration of the selenium-containing material may be selected by those skilled in the art without limitation. For example, it may be 0.1 nM to 10 μM, 1 nM to 9 μM, 10 nM to 5 μM, 100 nM to 4 μM, or 1 μM to 3 μM based on the total pharmaceutical composition. Further, when the pharmaceutical composition is a solid dosage form, the concentration of the selenium-containing material based on the total pharmaceutical composition may be 1 to 2,000 ppm, 10 to 1,500 ppm, 50 to 1,200 ppm, 100 to 1,000 ppm, or 200 to 800 ppm. However, it is not limited thereto.

In the present invention, the concentration of the antioxidant may be selected by those skilled in the art without limitation. For example, it may be 1 μM to 10 mM, 5 μM to 5 mM, 10 μM to 1 mM, 50 μM to 500 μM, 100 μM to 500 μM, or 200 μM to 400 μM based on the total pharmaceutical composition. Further, when the pharmaceutical composition is a solid dosage form, the concentration of the antioxidant based on the total pharmaceutical composition may be 1,000 to 500,000 ppm, 5,000 to 300,000 ppm, 10,000 to 200,000 ppm, or 20,000 to 100,000 ppm. However, it is not limited thereto.

In the present invention, the concentration of the selenium-containing material or the concentration of the antioxidant can be concentrated or diluted for use according to the purpose. For example, in consideration of the frequency of injection or intake of the pharmaceutical composition, the age, weight or gender of the subject to use the same, eating habits, the presence or absence of underlying diseases, and the like, it may be changed by those skilled in the art.

Further, the present invention relates to a health functional food for preventing or improving osteoarthritis, which includes a selenium-containing material and an antioxidant.

In the present invention, the health functional food may protect cartilage by maximizing the oxidative stress inhibitory effect in the cells constituting the cartilage based on the above-described mechanism, briefly, synergy between the selenium-containing material and the antioxidant. Alternatively, osteoarthritis can be prevented or improved.

In the health functional food of the present invention, the selenium-containing material may be included regardless of its type or chemical formula. For example, the material may be selenite, selenate or selenomethionine (SeMet), or a pharmaceutically acceptable salt thereof, but it is not limited thereto.

In the health functional food of the present invention, the antioxidant may be included without limitation to its type or chemical formula as long as it inhibits oxidative stress. For example, it may inhibit oxidative stress by suppressing reactive oxygen species (ROS) such as superoxide, hydrogen peroxide, and hydroxyl radical, and more specifically, may be N-acetylcysteine, ascorbic acid or glutathione, but it is not limited thereto.

The concentration of the selenium-containing material in the health functional food of the present invention can be selected by those skilled in the art without limitation. For example, it may be 0.1 nM to 10 μM, 1 nM to 9 μM, 10 nM to 5 μM, 100 nM to 4 μM, or 1 μM to 3 μM based on the total pharmaceutical composition. Further, when the health functional food is a solid formulation, the concentration of the selenium-containing material based on the total composition is 1 to 2,000 ppm, 10 to 1,500 ppm, 50 to 1,200 ppm, 100 to 1,000 ppm, or 200 to 800 ppm. However, it is not limited thereto.

The concentration of the antioxidant in the health functional food of the present invention may be selected by those skilled in the art without limitation. For example, it may be 1 μM to 10 mM, 5 μM to 5 mM, 10 μM to 1 mM, 50 μM to 500 μM, 100 μM to 500 μM, or 200 μM to 400 μM based on the total pharmaceutical composition. Further, when the health functional food is a solid formulation, the concentration of the antioxidant based on the total composition may be 1,000 to 500,000 ppm, 5,000 to 300,000 ppm, 10,000 to 200,000 ppm, or 20,000 to 100,000 ppm, however, it is not limited thereto.

In the present invention, the concentration of the selenium-containing material or the concentration of the antioxidant may be concentrated or diluted for use according to the purpose. For example, in consideration of the frequency of injection or intake of the health functional food, the age, weight or gender of the subject to use the same, eating habits, the presence or absence of underlying disease, and the like, it may be changed by those skilled in the art.

The pharmaceutical composition or health functional food of the present invention may further include a pharmaceutically acceptable carrier, and may be formulated along with such a carrier. As used herein, the term “pharmaceutically acceptable carrier” refers to a carrier or diluent that does not stimulate the organism and does not inhibit biological activities and properties of the administered compound. Pharmaceutical carriers acceptable in the composition formulated as a liquid solution are sterile and biocompatible, and may include saline, sterile water, Ringer's solution, buffered saline, albumin injectable solutions, dextrose solution, maltodextrin solution, glycerol, ethanol, and a mixture of one or more of these components. Further, if necessary, other typical additives such as antioxidants, buffers and bacteriostatic agents may be added. Diluents, dispersants, surfactants, binders and lubricants may also be added to formulate the pharmaceutical composition into injectable formulations, pills, capsules, granules or tablets such as aqueous solutions, suspensions, emulsions and the like.

The pharmaceutical composition or health functional food of the present invention is applicable in a form of any formulation containing the composition of the present invention as an active ingredient, and may be prepared in oral or parenteral formulations. The pharmaceutical formulations of the present invention may include forms suitable for oral, rectal, nasal, topical (including the cheek and sublingual), subcutaneous, vaginal or parenteral (intramuscular, subcutaneous) administration. Alternatively, forms suitable for administration by inhalation or insufflations may also be included.

The pharmaceutical composition or health functional food of the present invention is administered in a pharmaceutically effective amount. Effective dose levels may be determined depending on types or severity of disease of the patient, activity of drug, sensitivity to drug, administration time, administration route and rate of release, duration of treatment, factors including concurrent medications, and other factors well known in the medical field. The pharmaceutical composition of the present invention may be administered as an individual therapeutic agent or in combination with other therapeutic agents, may be administered sequentially or simultaneously with conventional therapeutic agents, and may be administered in single or multiple doses. Taking all of the above factors into consideration, it is important to administer the pharmaceutical composition in an amount that can achieve maximum effects with a minimum amount without side effects, which may be easily determined by those skilled in the art.

The dosage of the pharmaceutical composition or health functional food may vary widely depending on the weight, age, gender, health conditions or diet of a patient, administration time, administration method, excretion rate and severity of the disease, and the appropriate dosage may depend on, for example, an amount of drug accumulated in the patient's body and/or specific efficacy of the used drugs. For example, it may be 0.01 μg to 1 g per kg of body weight. Further, the pharmaceutical composition of the present invention may be administered once or several times per unit time during unit periods of time such as daily, weekly, monthly or yearly, or may be continuously administered using an infusion pump for a long time. The number of repeated administration doses is determined in consideration of a residential time of drug in the body, a drug concentration in the body, etc. Even after recovery following the drug treatment, the composition may be further administered to prevent recurrence, i.e., relapse of the disease.

Hereinafter, the present invention will be described in detail with reference to the following examples.

### Experimental Example 1. Collection of Human Tissue Samples

Human osteoarthritis specimens were obtained from osteoarthritis patients who underwent total knee arthroplasty at Seoul National University (SNU) Boramae Medical Center. Seoul National University Boramae Hospital (Institutional Review Board) approved the collection of human biological materials (IRB No. 30-2017-48), and Seoul National University IRB approved the use of these materials (IRB No. E1803/003-009). From all subjects, written informed consents were obtained prior to total knee arthroplasty surgery. In the present invention, only samples obtained from female patients were used, and human cartilage tissue sections were stained with alcian blue and graded according to the OARSI grade system.

### Experimental Example 2. Management of Laboratory Animals

All animal experiments were approved by the Seoul National University Animal Care Committee (IACUC No. SNU-151202-6, SNU-190919-6, SNU-190910-1, SNU-191115-3), and the design, analysis and reporting of animal experiments were performed according to the Animal Under Study: In Vivo Experiment Reporting Guidelines, and the mice were bred in the specific pathogen free (SPF) animal facility at Seoul National University. Animals were maintained under constant temperature and humidity with a controlled light/dark cycle (12:12 h).

In order to secure the SEPHS1 knockout mouse line, floxed SEPHS1 (SEPHS1^{fl/fl}) mice were prepared and crossed with Col2a1-Cre or Col2a1-CreER^{T2} mice.

To secure an aging-associated spontaneous osteoarthritis model, 12-month-old male SEPHS1^{fl/fl} or SEPHS1^{fl/fl}; Col2a1-CreER^{T2} mice were injected intraperitoneally with 80 μg/g body weight of TMX dissolved in corn oil (Sigma Aldrich) daily for 5 consecutive days. It was confirmed by PCR that the Sephsl gene was ablated by cre expressed in knee articular cartilage of 12-week-old Sephs1^{fl/fl} mice injected with TMX. Tissue Genomic DNA extraction SV mini prep kit (MG MED) was used to extract genomic DNA from paraffinized chondrocytes derived from knee articular cartilage.

### Experimental Example 3. Diet Control in Mice

Selenium diet control in mice was performed as follows. 12-week-old male SEPHS1^{fl/fl} or SEPHS1^{fl/fl}; Col2a1-CreER^{T2} mice were injected with 80 μg/g body weight of TMX for 5 consecutive days, thereafter, 5 days prior to Sham or DMM surgery, the animals were fed with either a selenium-deficient (SeD) torula yeast-based diet (0.026 μg/g Se) or an adequate selenium-containing diet (0.4 μg/g Se). Mice were fed with the same diet for an additional 6 weeks until sacrifice after surgery.

Selenate supplementation or NAC treatment was performed by supplying mice with drinking water in which sodium selenate (1 ppm) or NAC (1 mg/mL) was dissolved. Further, to Sephs1-iCKO mice, NAC, selenate or vehicle was supplemented 5 days prior to DMM surgery.

### Experimental Example 4. Induction of Osteoarthritis in Mice

Induction of age-related spontaneous osteoarthritis in Sephs1-iCKO mice was performed in 18-month-old mice. 12-month-old SEPHS1^{fl/fl} or SEPHS1^{fl/fl}; Col2a1-CreER^{T2} mice were underwent with 5 consecutive intraperitoneal TMX injections and sacrificed at 18 months of age.

Posttraumatic osteoarthritis (OA) was induced by executing DMM surgery in 12-week-old (WT C57BL/6) or 13-week-old (SEPHS1^{fl/fl} or SEPHS1^{fl/fl}; Col2a1-CreER^{T2}) mice, and mice (sham) without DMM after incision were used as a control. These mice were sacrificed 6 or 8 weeks after DMM or sham surgery.

As an aging-associated OA model, 24-month-old C57BL/6 male mice were used for histological analysis and 3-month-old mice were used as a control. The extent of cartilage degeneration in the knee joint was assessed by safranin-O staining and scored using the OARSI grading system.

### Experimental Example 5. Cell Culture

For primary culture of mouse articular chondrocytes, cells derived from femoral condyle and tibial plateaus of 5-day-old ICR mice were isolated. Chondrocytes were cultured in DMEM medium containing 10% FBS (Gibco), 100 units/mL of penicillin, and 100 μg/mL of streptomycin. After 2 days, cells were treated according to the purpose of each experiment. The cultured cells were maintained at 37° C. under 5% CO₂ and 3% O₂ conditions.

### Example 1. Confirmation of Selenoprotein Expression in Osteoarthritis-Induced Human or Mouse Chondrocytes

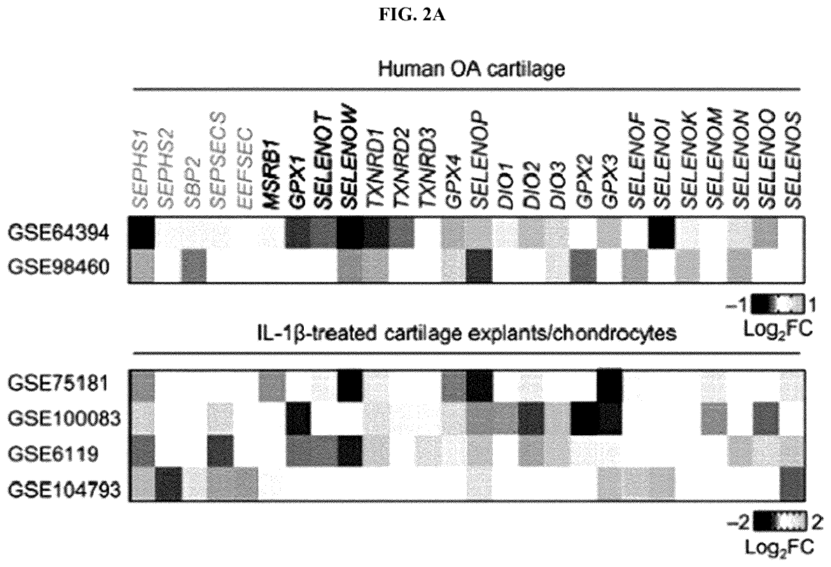

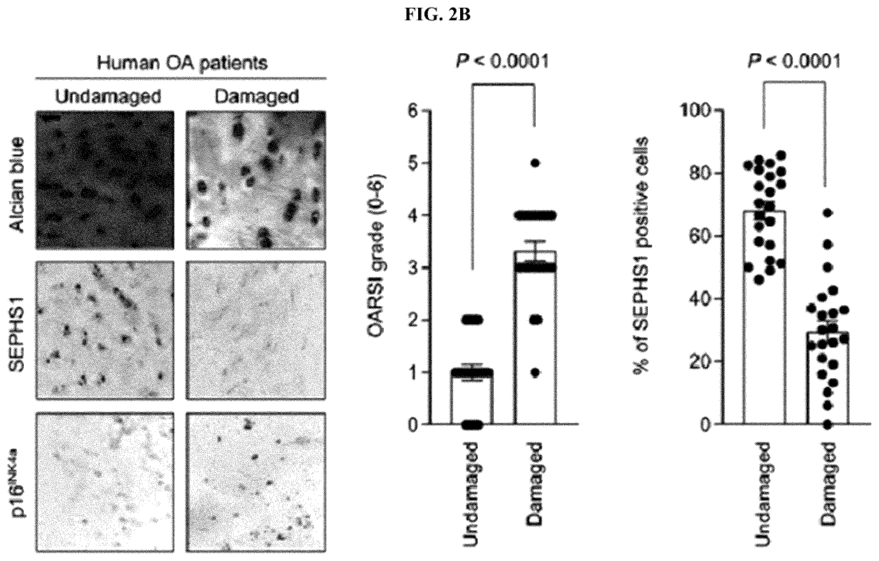

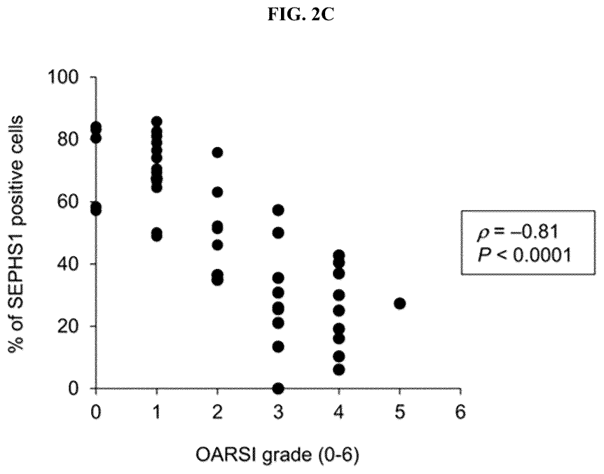

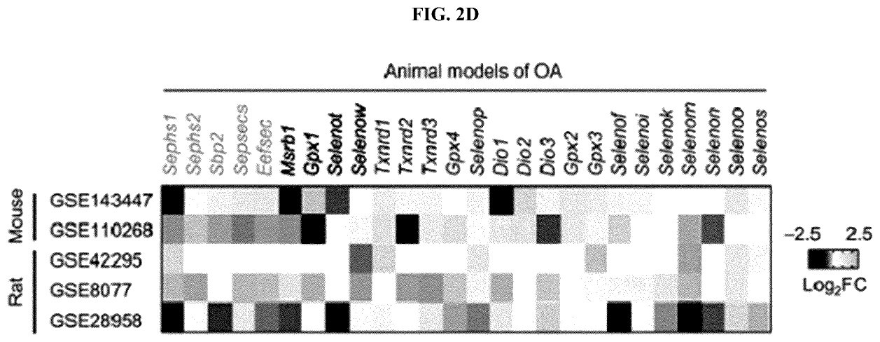

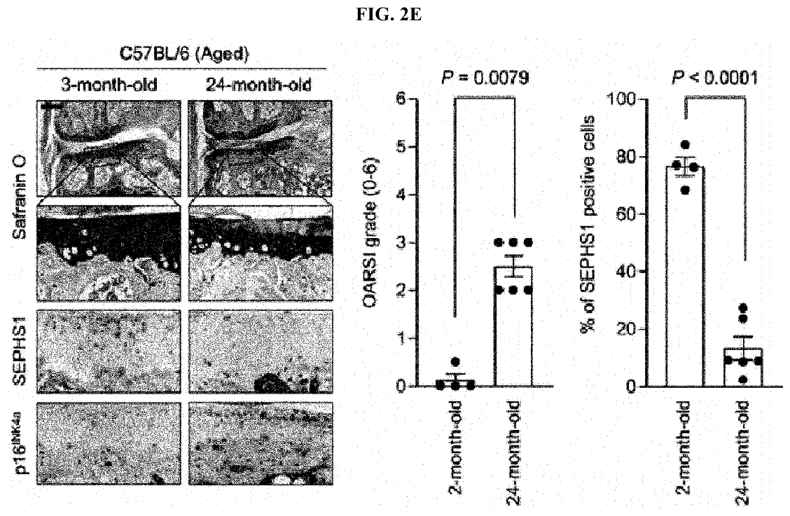

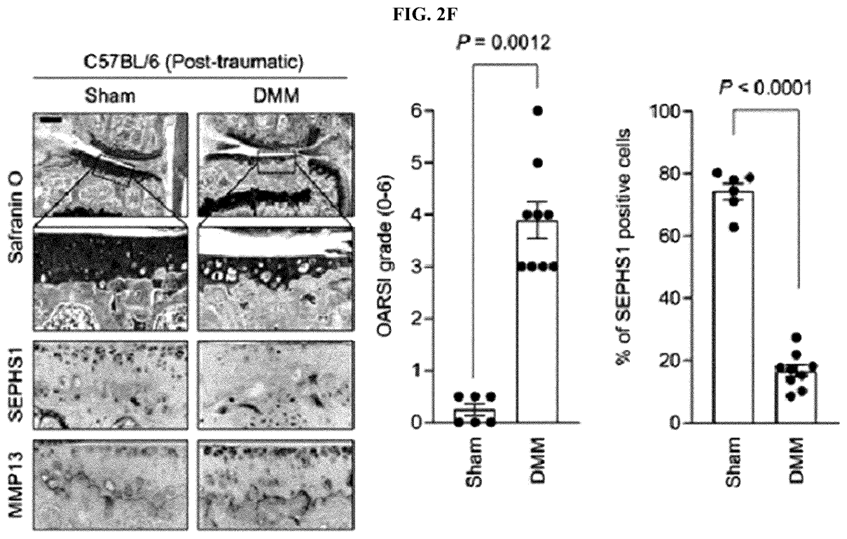

To identify regulatory mechanism of selenium metabolism in osteoarthritis conditions, transcriptomes were analyzed around genes involved in selenium metabolism regulation mechanisms (SEPHS1, SEPHS2, SBP2, SEPSECS, and EEFSEC), and selenoproteins (MSRB1, GPX1, SELENOT, SELENOW, TXNRDI, TXNRD2, TXNRD3, GPX4, SELENOP, DIOI, DIO2, DIO3, GPX2, GPX3, SELENOF, SELENOI, SELENOK, SELENOM, SELENON, SELENOO, SELENOS) through Gene Expression Omnibus (GEO). Among the corresponding regulators, it was confirmed that SEPHS1 was down-regulated in the transcriptome of patients with osteoarthritis, which was similar to the results in cartilage treated with IL-1β. It was further confirmed that stress-related selenoproteins (MSRB1, GPX1, SELENOT, SELENOW) were down-regulated in the cartilage of human OA patients. Although SEPHS1 was sufficiently expressed in the cartilage tissue part with less damage from joints of patients with osteoarthritis, it was confirmed that SEPHS1 was hardly observed in the severe part. Further, it was confirmed that the expression of SEPHS1 has a strong negative correlation with OARSI grade (FIGS. 2A to 2D).

It was confirmed that the amount of SEPHS1 transcription and stress-related selenoproteins were also reduced in an osteoarthritis animal model. In addition, it was confirmed that the expression level of SEPHS1 was strongly suppressed in the knee osteoarthritis model of aged mice. Further, in the case where post-traumatic osteoarthritis was induced through DMM (destabilization of medial meniscus) surgery, it was also confirmed that the expression of SEPHS1 was strongly suppressed (FIGS. 2E and 2F).

### Example 2. Confirmation of Antioxidant Capacity Control Ability and Aging Control Ability Associated with Selenoprotein Deficiency in Chondrocytes

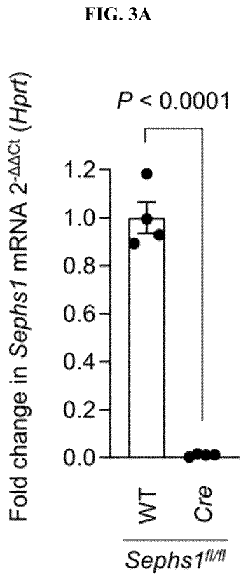

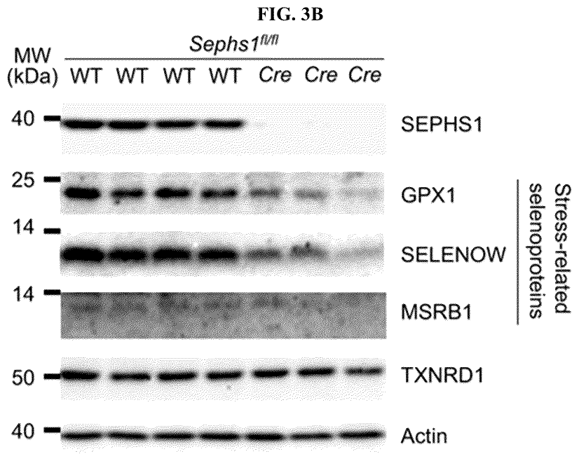

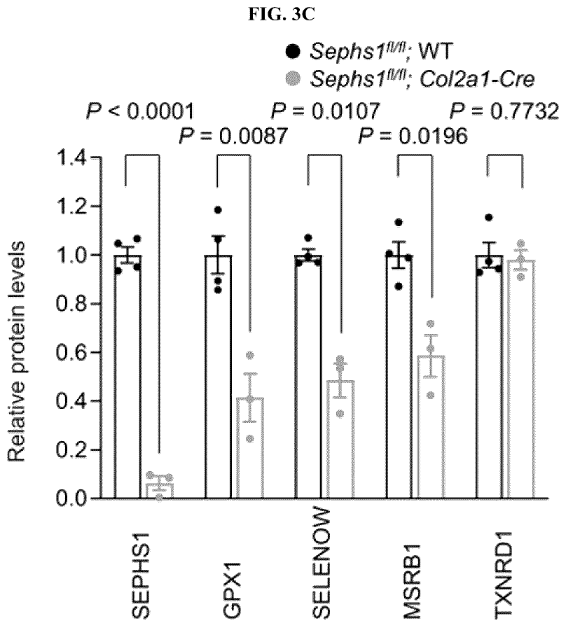

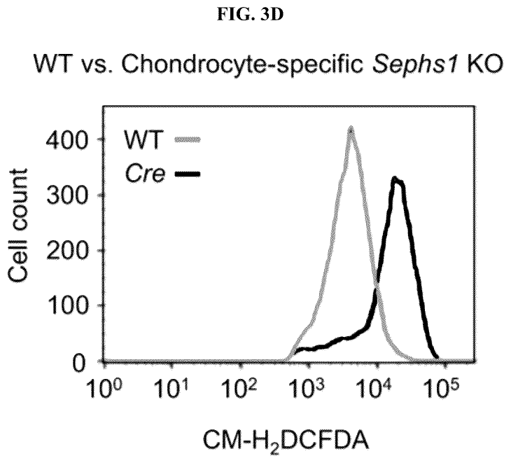

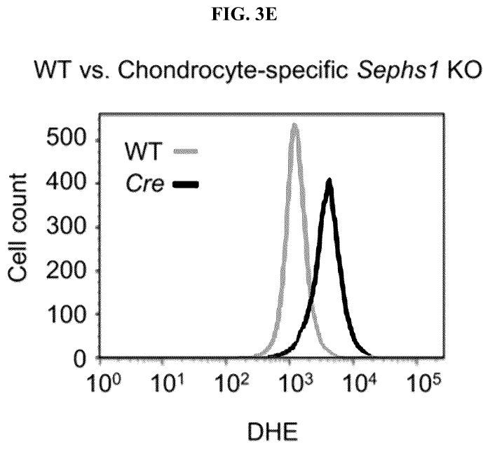

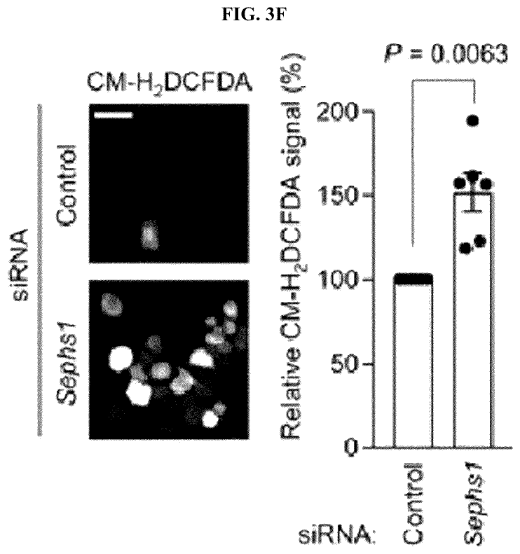

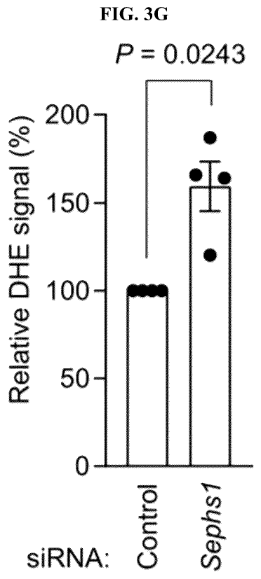

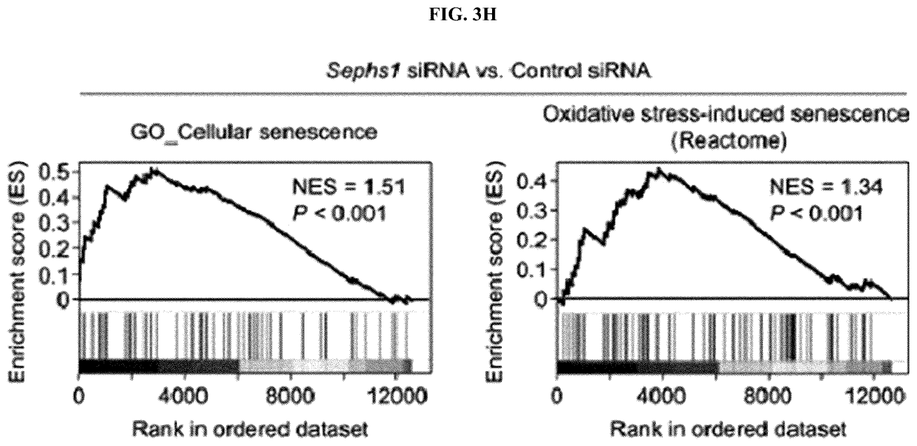

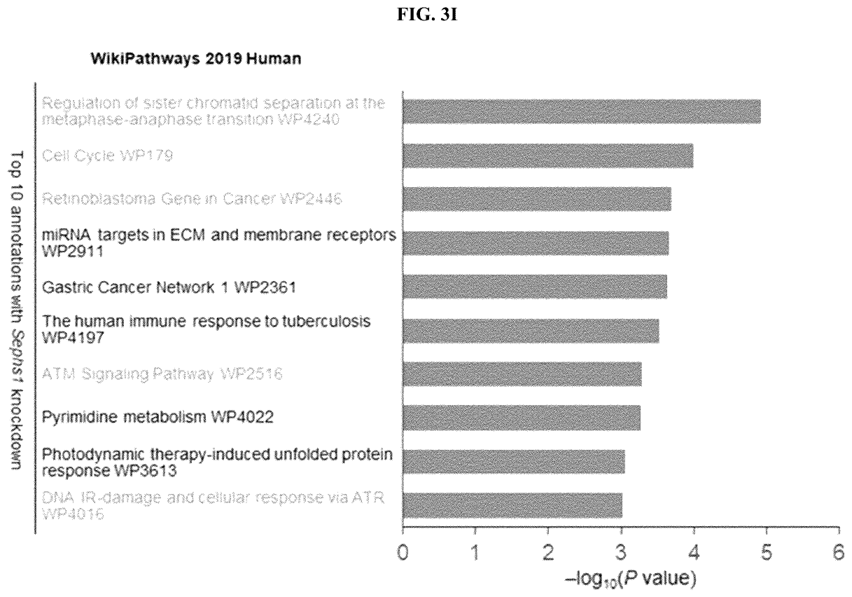

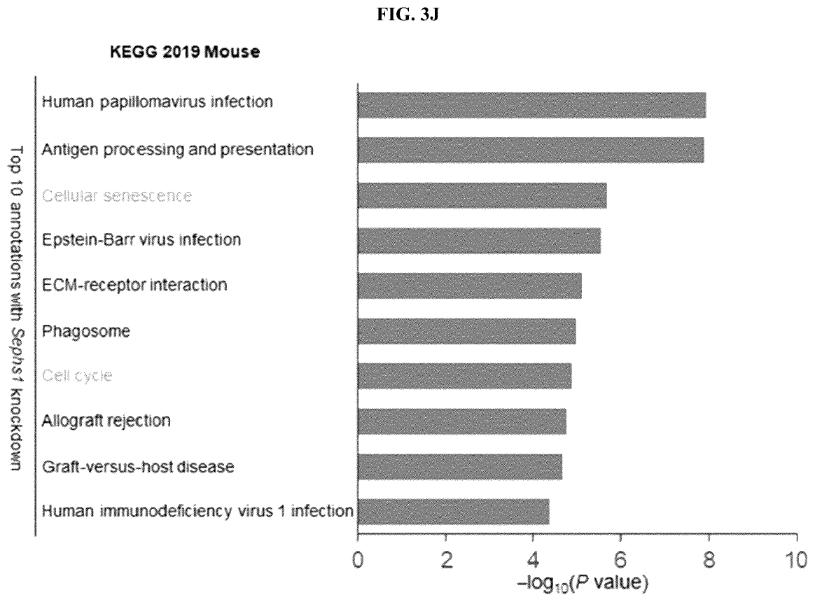

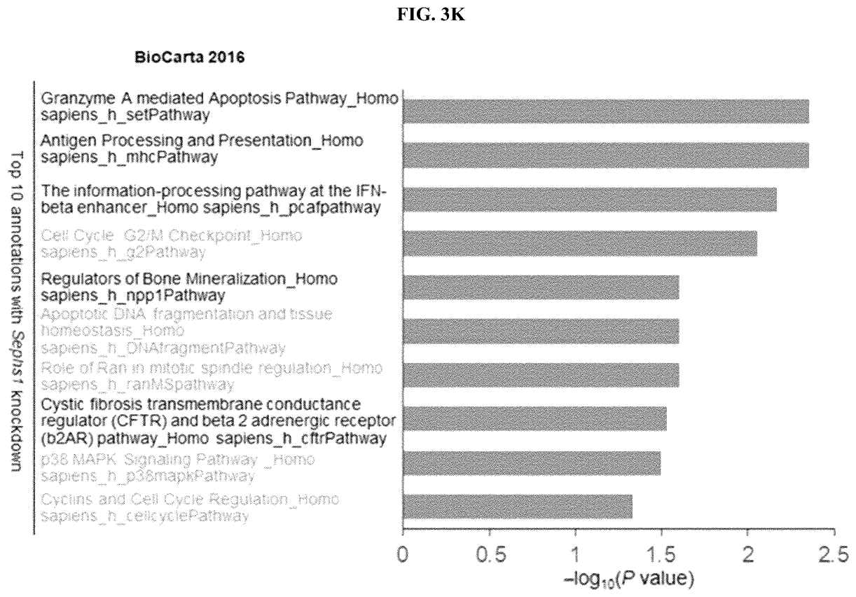

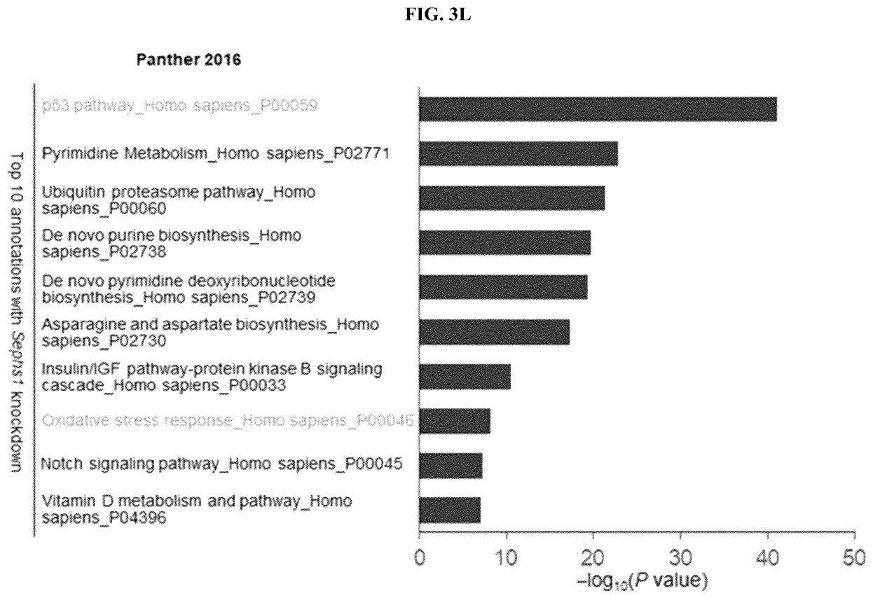

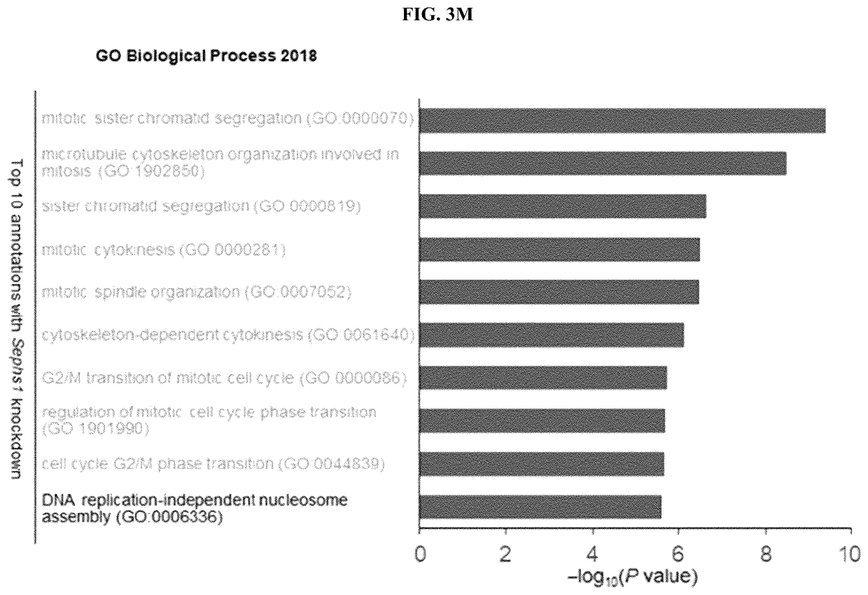

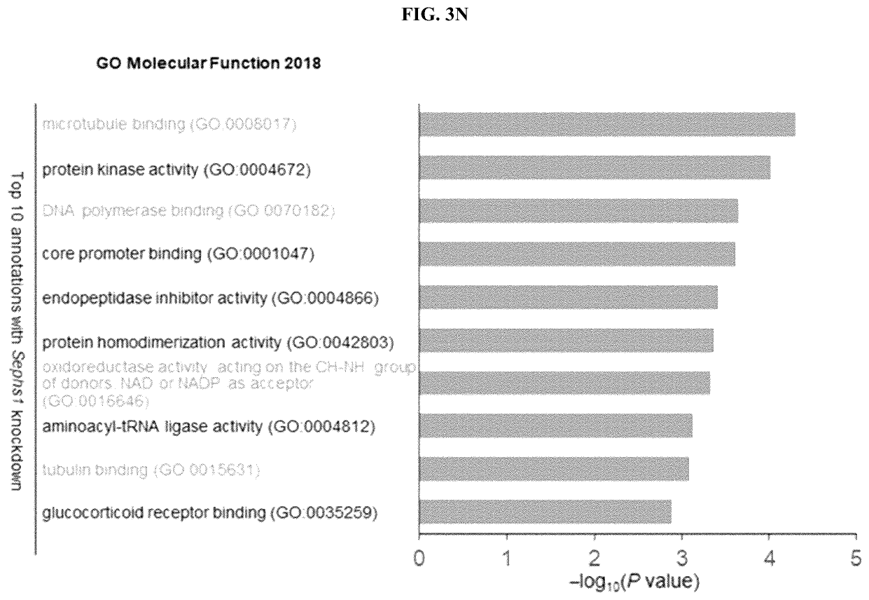

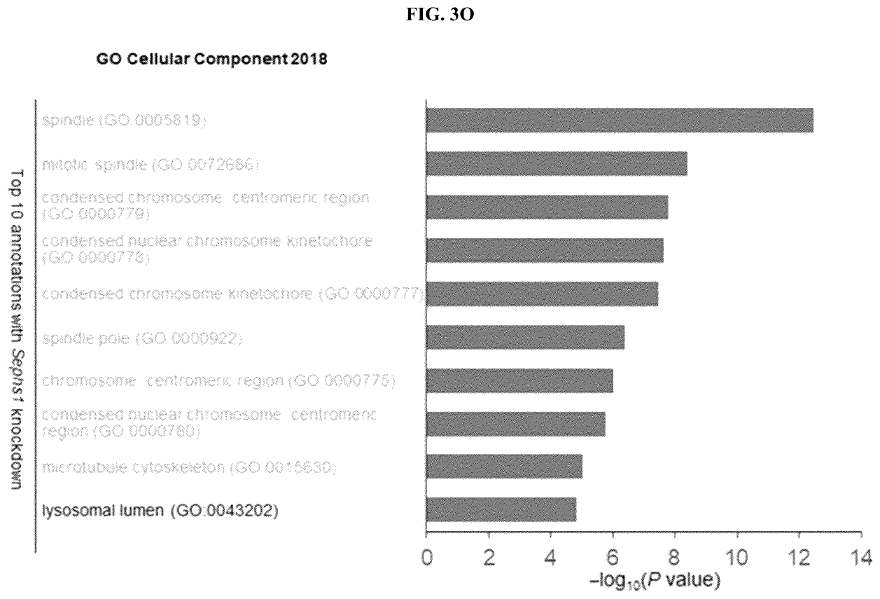

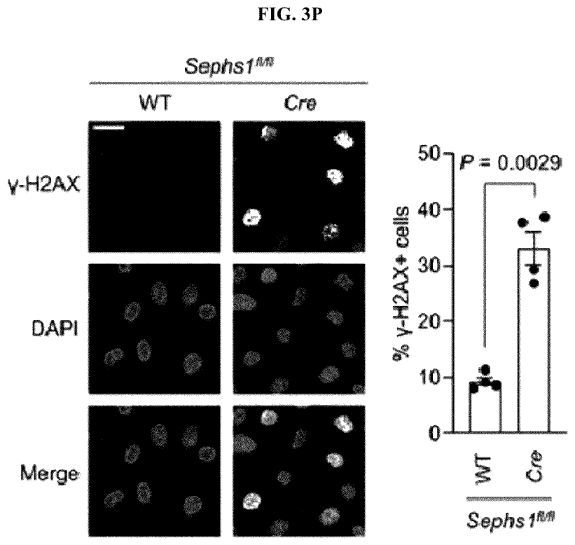

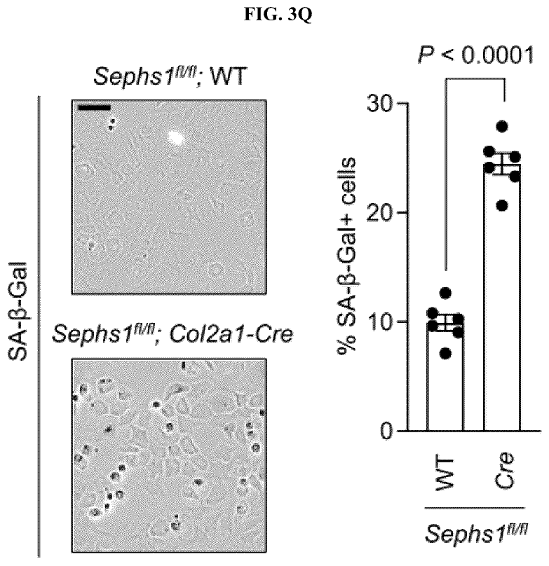

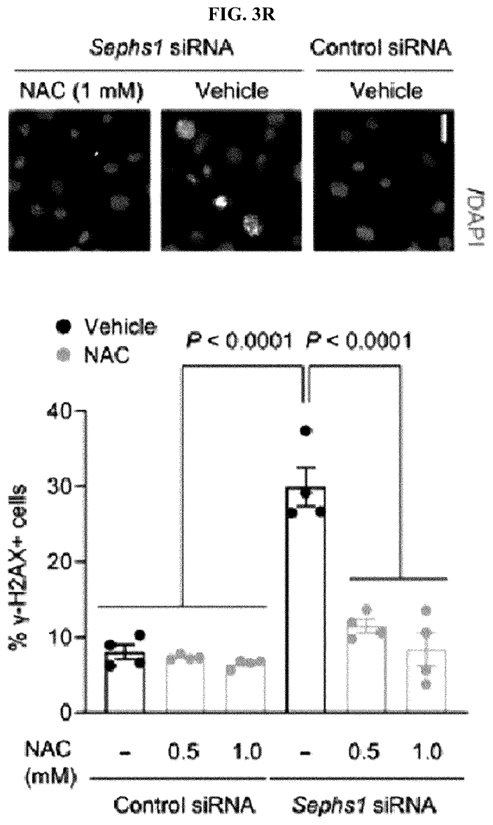

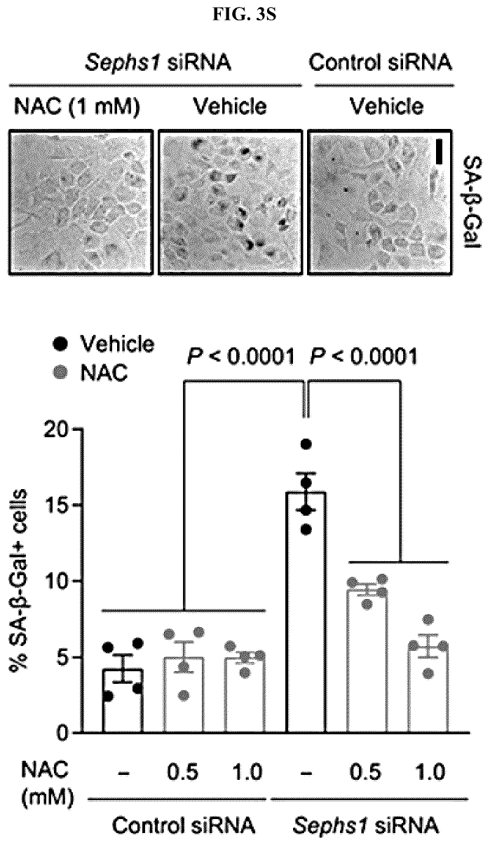

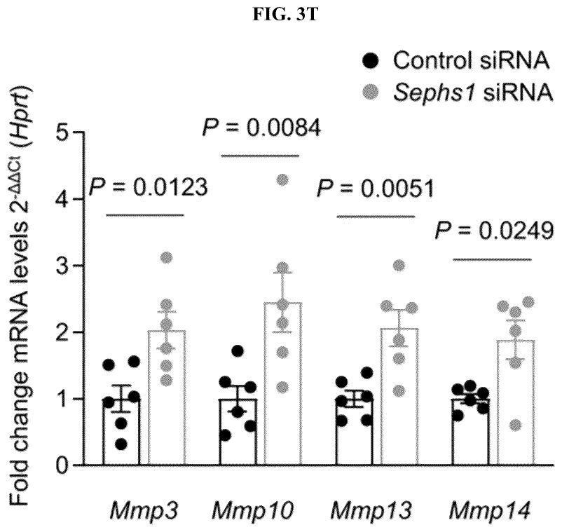

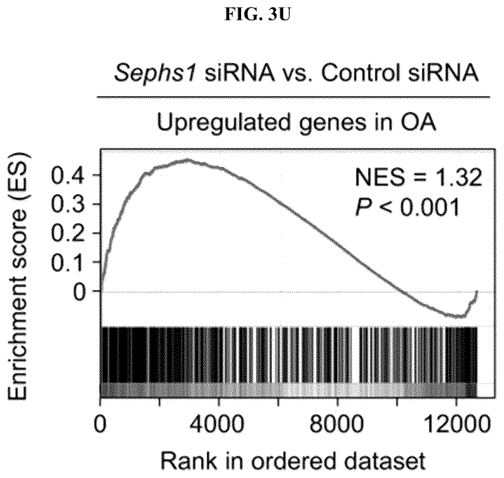

In order to confirm the regulatory mechanism of stress-related selenoproteins' expression by SEPHS1 reduction, cartilage-specific conditional knockout mice (SEPHS1^{fl/fl}: Col2a1-Cre, hereinafter referred to as SEPHS1 cKO mice) were constructed. It was confirmed that both the transcript and protein of SEPHS1 were hardly observed in chondrocytes derived from SEPHS1 cKO mice, and it was further confirmed that the expression of stress-related selenoproteins (GPX1, SELENOW and MSRB1) was reduced in chondrocytes in which SEPHS1 is knocked out. On the other hand, it was confirmed that the expression of housekeeping selenoprotein thioredoxin reductase 1 (TXNRD1) was not changed (FIGS. 3A to 3C).

In order to confirm the relationship between regulation of stress-related selenoproteins by SEPHS1 and antioxidant functions, the amount of reactive oxygen species (ROS) was determined in SEPHS1 knockout mice. The amounts of H₂O₂ and O₂⁻ in cells were measured, respectively, using CM-H2DCFDA and dihydroethidium (DHE). As a result, it was confirmed that ROS was significantly increased. Similarly, it was confirmed that ROS was increased even when Sephs1-specific siRNA was treated (FIGS. 3D to 3G).

Further, RNA was obtained from chondrocytes in which SEPHS1 was inhibited with siRNA and normal chondrocytes, and RNA sequencing was conducted, followed by performing Gene Ontology (GO) and pathway analysis. As a result, it was confirmed that gene groups related to DNA damage response, cell cycle arrest and oxidative stress response were strongly regulated in cartilage in which SEPHS1 was knocked down. According to gene set enrichment analysis (GSEA), it was confirmed that the dysregulated genes were strongly associated with cellular senescence or oxidative stress-induced senescence term (FIGS. 3H to 3O). These annotations have previously been closely related to “senescence,” which is an important cellular phenomenon that disrupts homeostasis during the development of osteoarthritis, and implied that the regulation of selenoproteins including SEPHS1 is significantly related to the regulation of aging.

In addition, DNA damage and senescence-associated β-galactosidase (SA-β-gal) activity were measured to determine the relationship between SEPHS1 deficiency and cellular senescence. In the chondrocytes derived from mice in which SEPHS1 was knocked out, it was confirmed that γ-H2AX foci were formed in the nuclear region, and γ-H2AX activity was significantly increased. In order to determine whether the DNA damage and the increase in SA-β-gal activity caused by SEPHS1 inhibition were caused by the accumulation of ROS, SEPHS1 knockout mouse-derived chondrocytes were additionally treated with N-acetylcysteine (NAC), followed by repeatedly performing the same experiment. As a result, it was confirmed that DNA damage caused by SEPHS1 inhibition and the increase in SA-β-gal activity were reduced after NAC treatment (FIGS. 3P to 3S).

Further, it was confirmed that the expression of various senescence-associated secretory phenotypes (SASPs) was increased by knockdown of SEPHS1. In addition, according to GSEA analysis, it was confirmed that the gene group overall increased in the cartilage from osteoarthritis patient group was equally increased by SEPHS1 knockdown (FIGS. 3T and 3U).

### Example 3. Osteoarthritis Phenotype Analysis According to Cartilage-Specific Conditional SEPHS1 Knockout

Aging-associated spontaneous osteoarthritis or post-traumatic osteoarthritis was induced by the method of Experimental Example 4 in mice in which SEPHS1 was specifically knocked out with tamoxifen (TMX) (SEPHS1^{fl/fl}; Col2alcreERT2, hereinafter referred to as SEPHS iCKO mice).

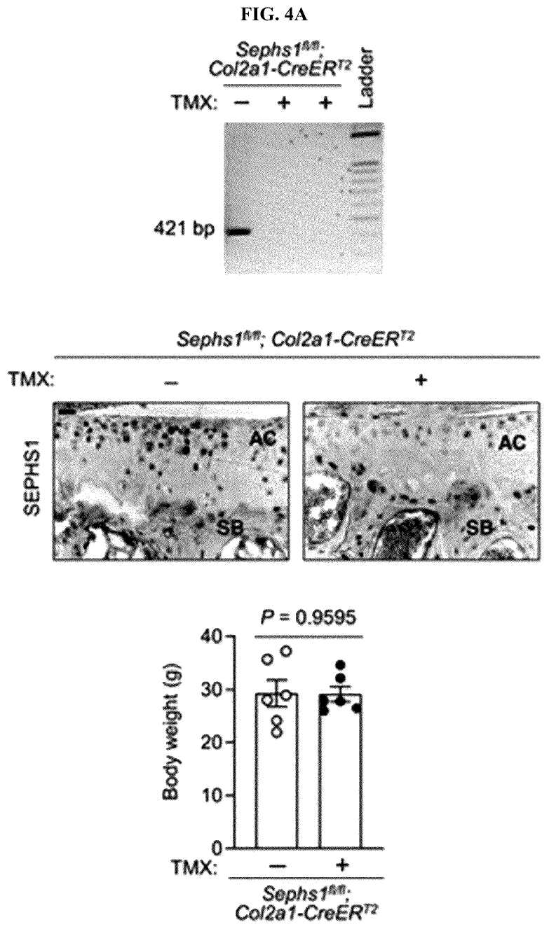

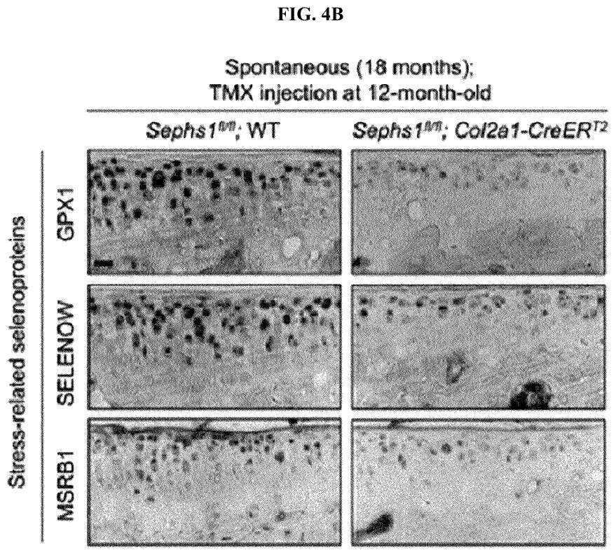

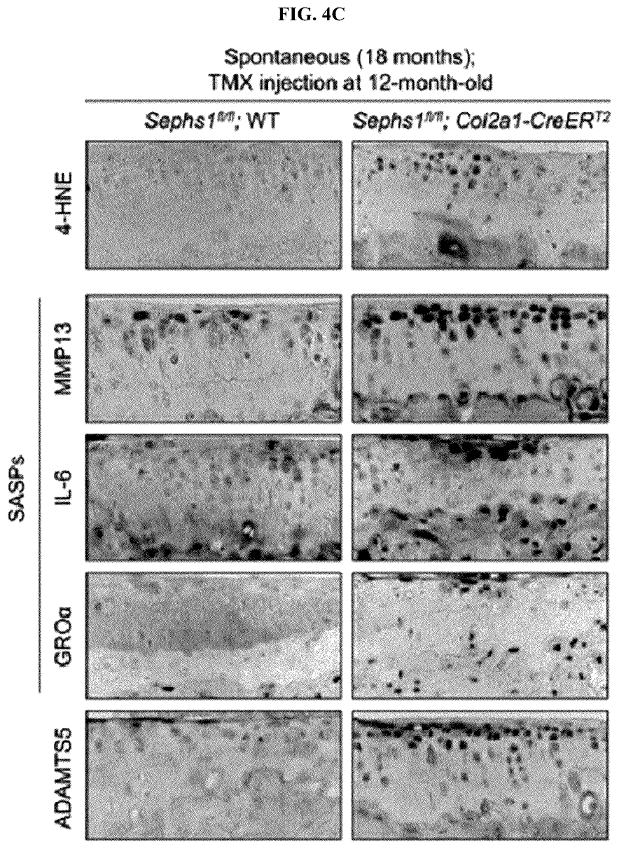

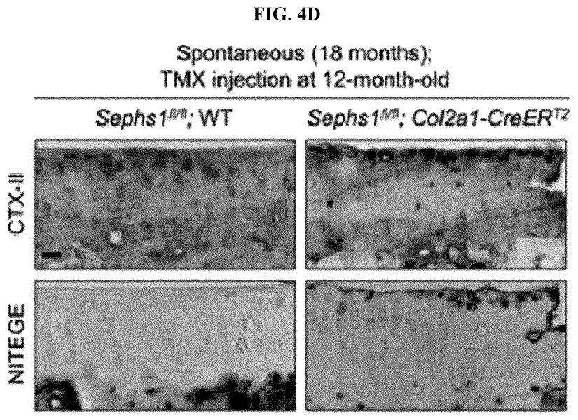

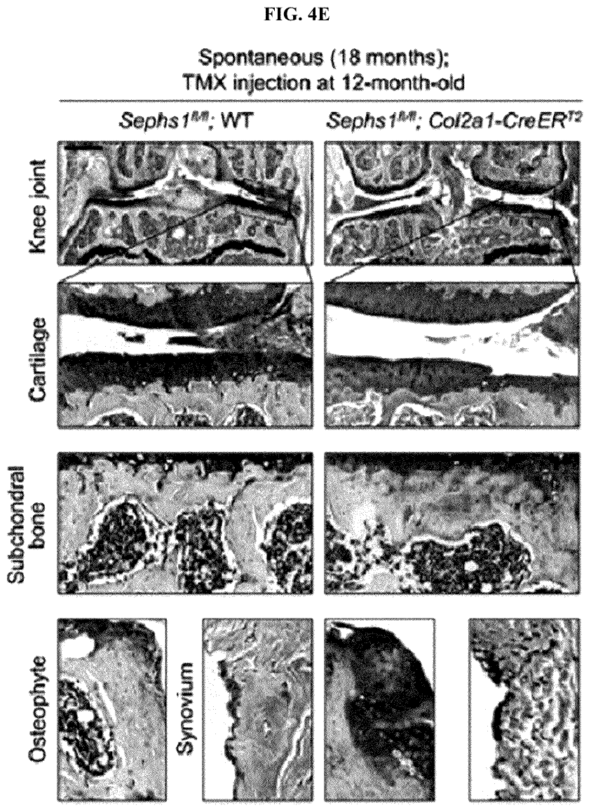

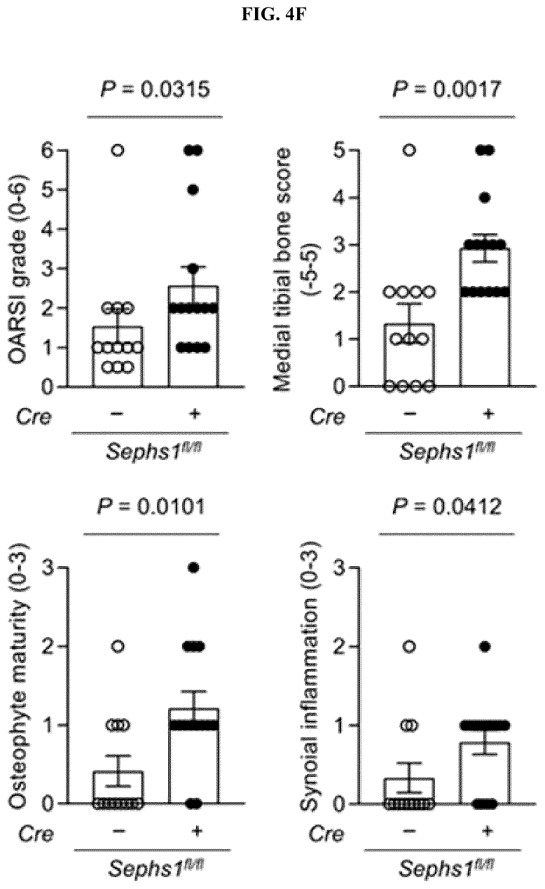

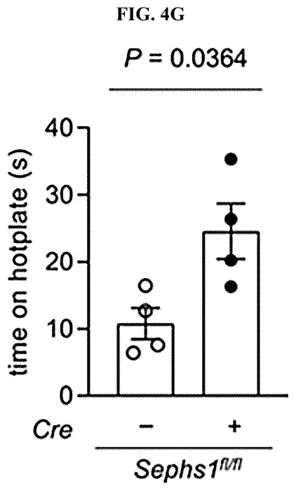

As a result, it was confirmed that 18-month-old SEPHS1 iCKO mice showed symptoms of osteoarthritis including subchondral bone sclerosis, osteophyte development, and synovitis, and exhibited decreased expression of stress-related selenoproteins including GPX1, SELENOW and MSRB1. At the same time, it was confirmed that an impairment in the regulation of redox homeostasis of articular cartilage occurred as illustrated by an increase in the amount of 4-hydroxynonenal (4-HNE). Furthermore, it was confirmed that the expression of inflammatory factors such as IL-6 or GROα was increased, and the expression of MMP13 and ADAMTS5, which are factors related to OA cartilage degeneration, was also increased. Further, as a behavioral test of sensory dysfunction, a response time was increased compared to the control in hot plate analysis, thereby confirming that knockout of SEPHS1 aggravates sensory impairment, which commonly occurs during the progression of osteoarthritis (FIGS. 4A to 4G).

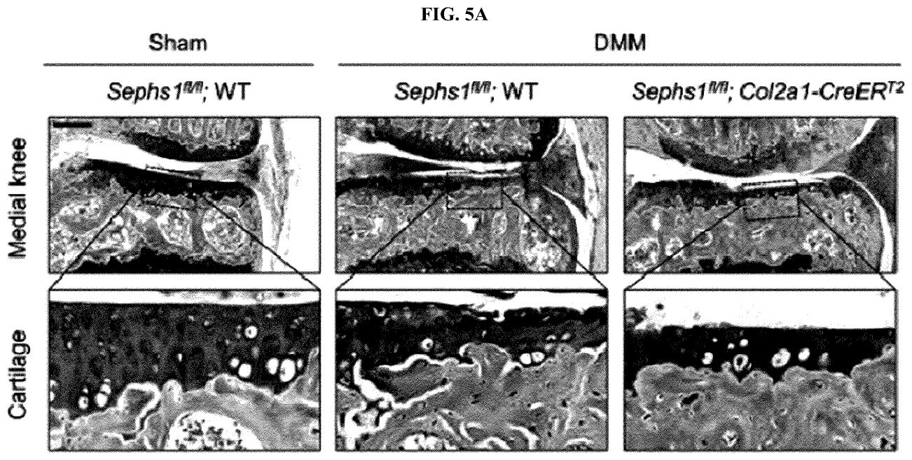

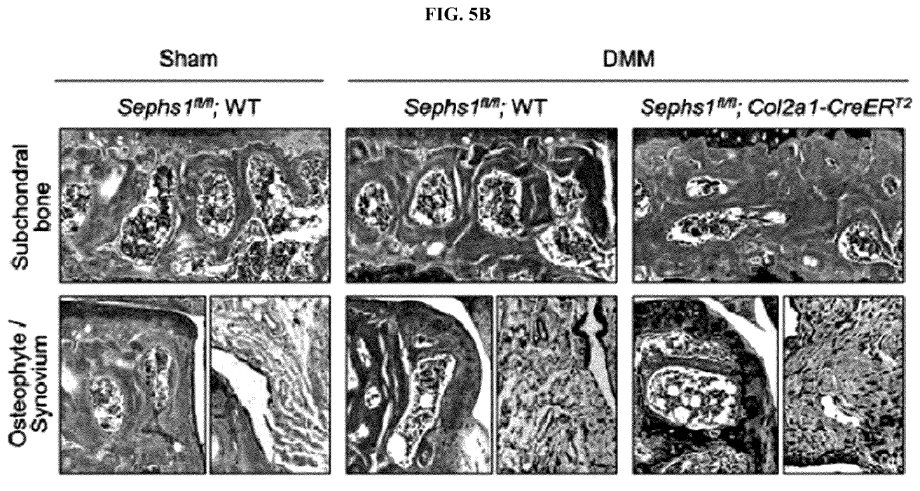

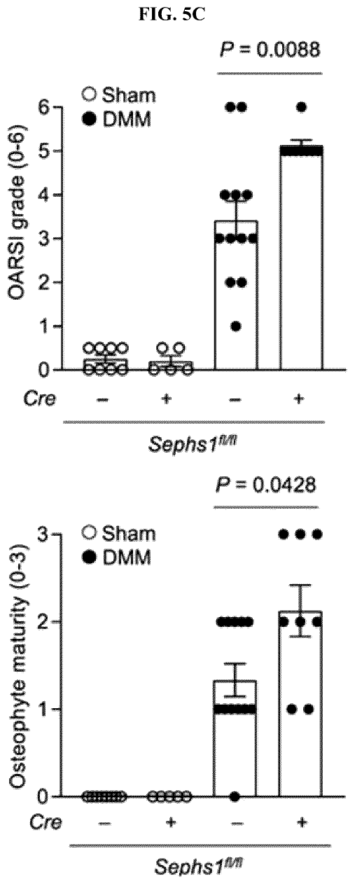

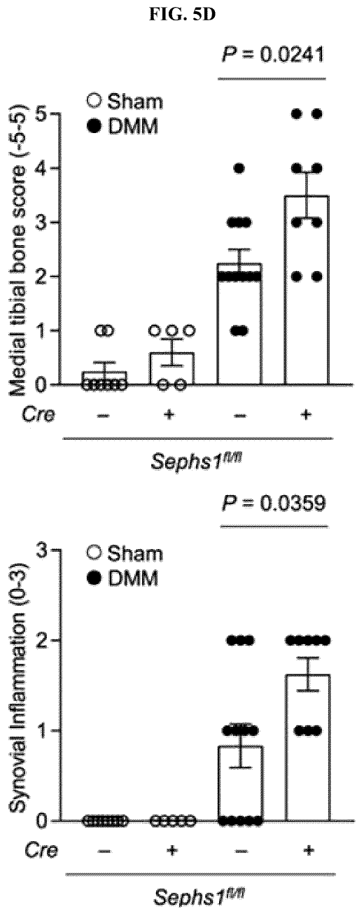

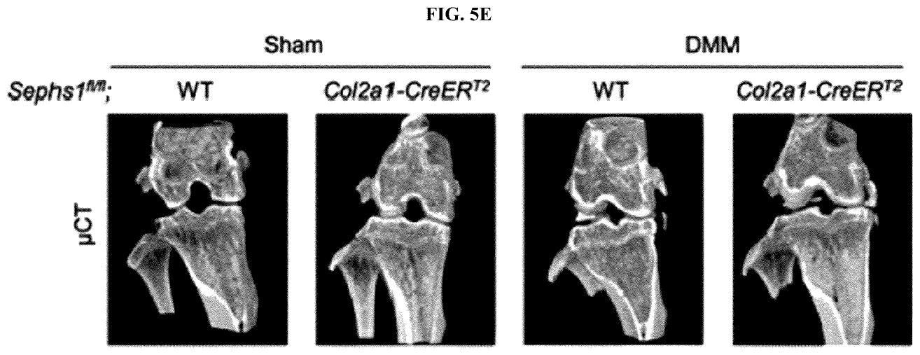

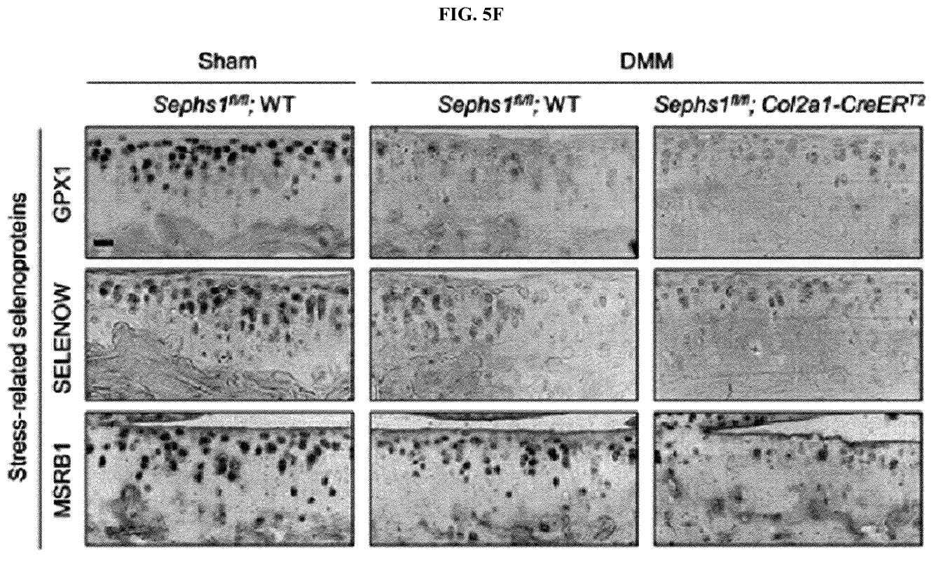

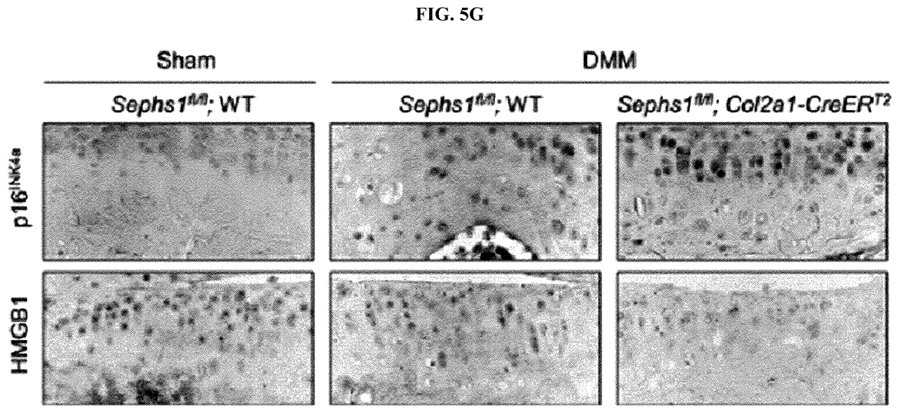

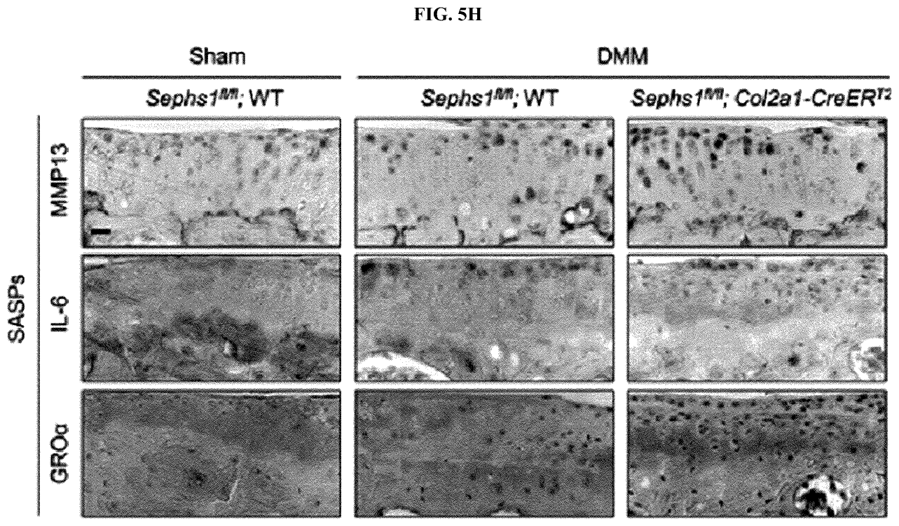

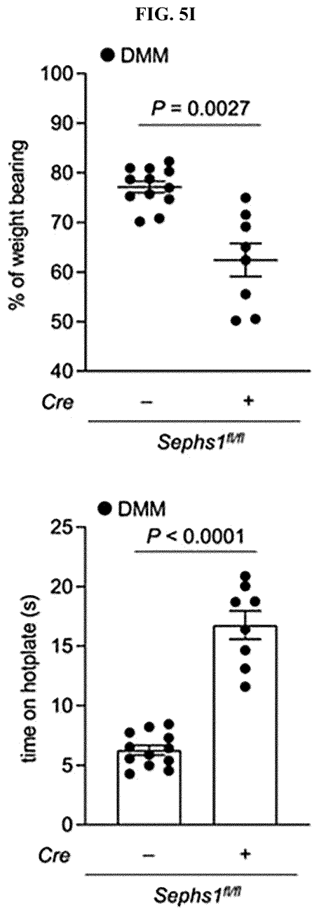

Furthermore, similar to aging-associated spontaneous osteoarthritis, it was also confirmed that SEPHS1 iCKO mice, in which post-traumatic osteoarthritis was induced through DMM, showed symptoms of osteoarthritis including subchondral bone sclerosis, osteophyte development and synovitis. In addition, microcomputed tomography (μCT) showed an increased occurrence of bone remodeling associated with osteoarthritis. Likewise, at the molecular level, it was confirmed that the aging marker p16^{INK4a} was increased and nuclear HMGBI was decreased by the knockout of SEPHS1. Further, it was confirmed that the expression of SASPs such as MMP13, IL-6 and GROα were increased thereby. In addition, it was confirmed that SEPHS1-iCKO mice felt more severe pain by determining the occurrence of weight imbalance, which is caused due to a difference in the weight-bearing ratio between the legs that had undergone DMM surgery and the other legs without operation. Similar to the model of aging-associated spontaneous osteoarthritis, it was confirmed that the response time was increased in the hot plate analysis (FIGS. 5A to 5I).

### Example 4. Analysis of Osteoarthritis Phenotype in SEPHS1 Knockout Mice According to Antioxidant or Selenate Treatment

In the previous post-traumatic osteoarthritis induction experiment, in order to determine whether the worsening of osteoarthritis symptoms by SEPHS1 deficiency was due to the increased oxidative stress, the osteoarthritis phenotype was investigated while supplying NAC as an antioxidant, as in Experimental Example 3. At the same time, as in Experimental Example 3, it was confirmed whether intake of selenate used as a substitute for selenium could restore the worsening of osteoarthritis due to SEPHS1 deficiency.

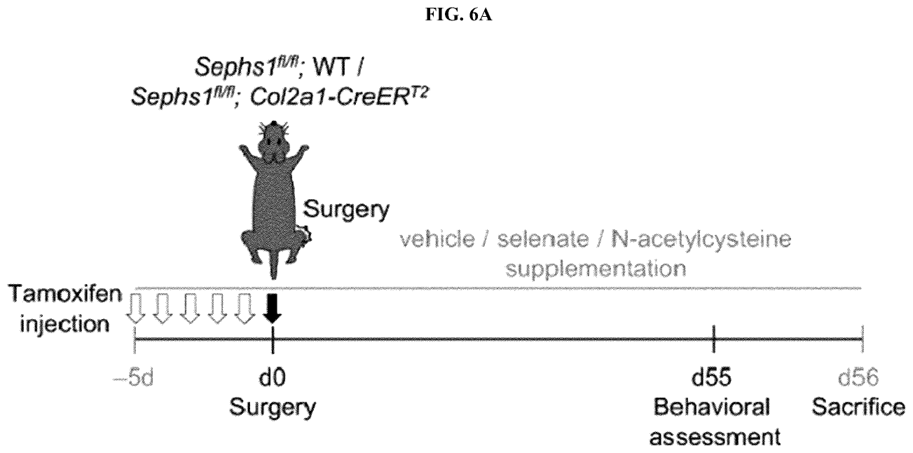

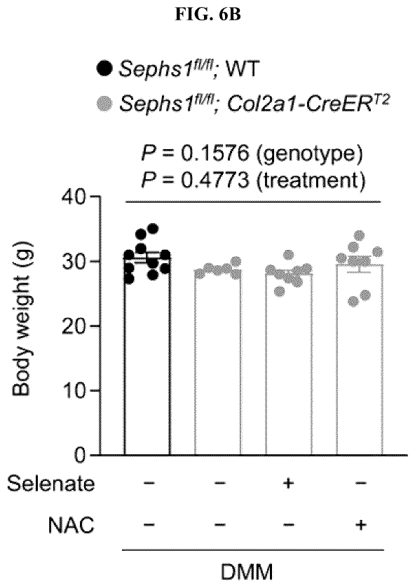

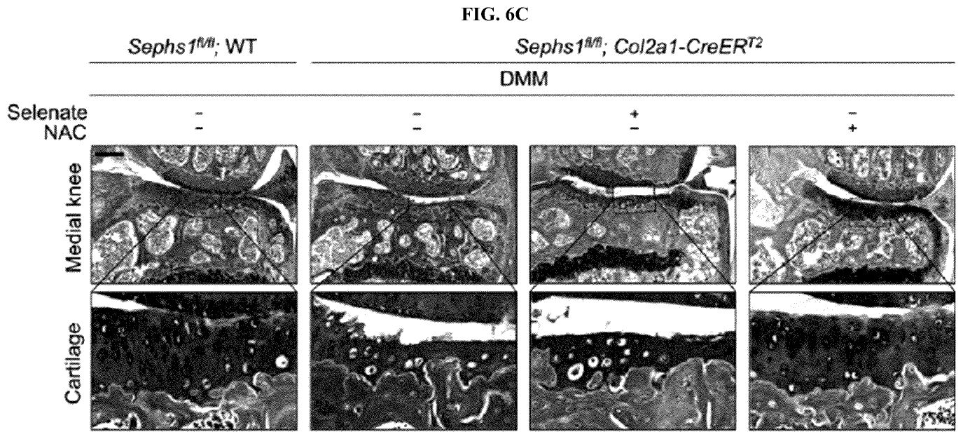

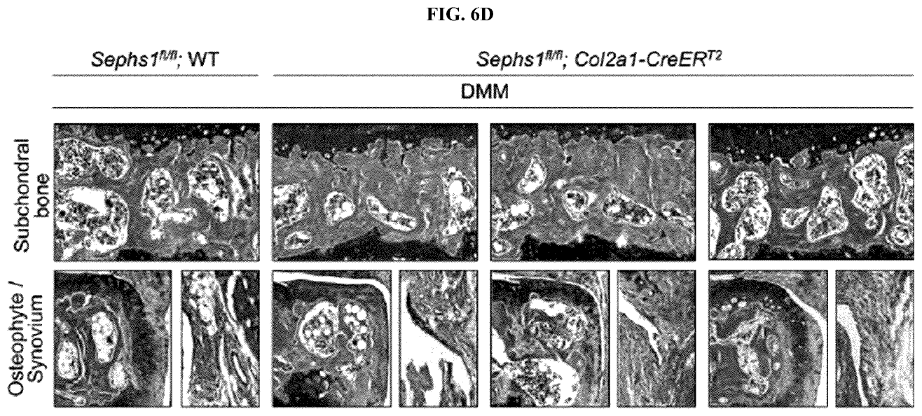

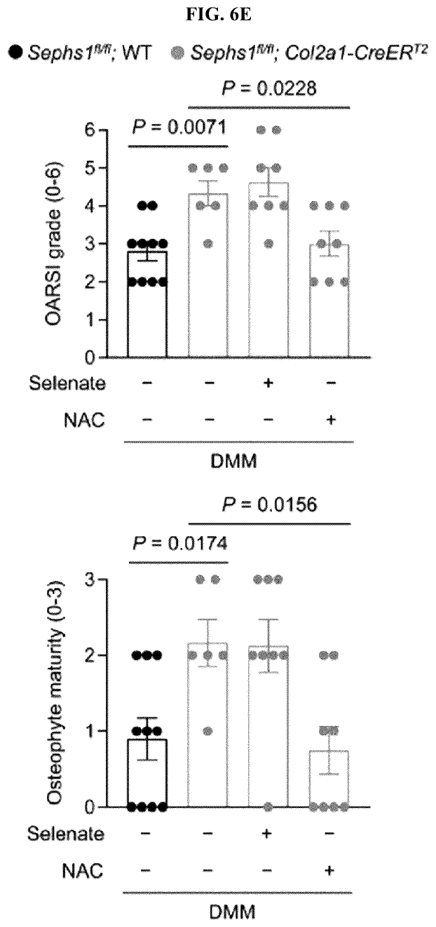

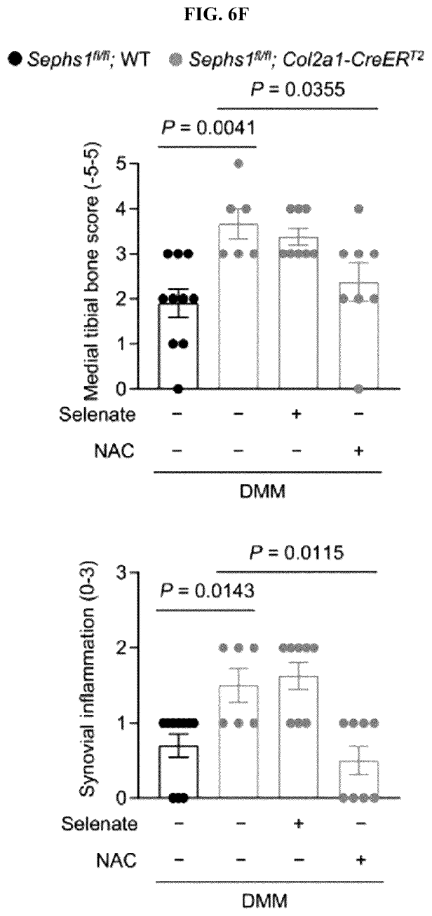

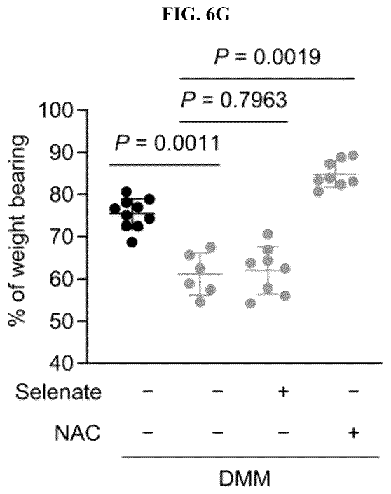

As a result, it was confirmed that there was no significant change in the body weight of the animals by the dietary control of selenium, and it was also confirmed that the osteoarthritis phenotype of the SEPHS1-iCKO mice was alleviated by the supply of NAC. At the same time, it was confirmed that the weight imbalance between the operated legs and the other legs without operation was alleviated. On the other hand, supplying selenate did not restore the osteoarthritic phenotype of SEPHS1-iCKO mice (FIGS. 6A to 6G). In summary, the oxidative stress increased by SEPHS1 deficiency may play a role in accelerating the worse of osteoarthritis.

### Example 5. Analysis of Osteoarthritis Phenotype of Mice According to Selenium Deficiency and SEPHS1 Knockout

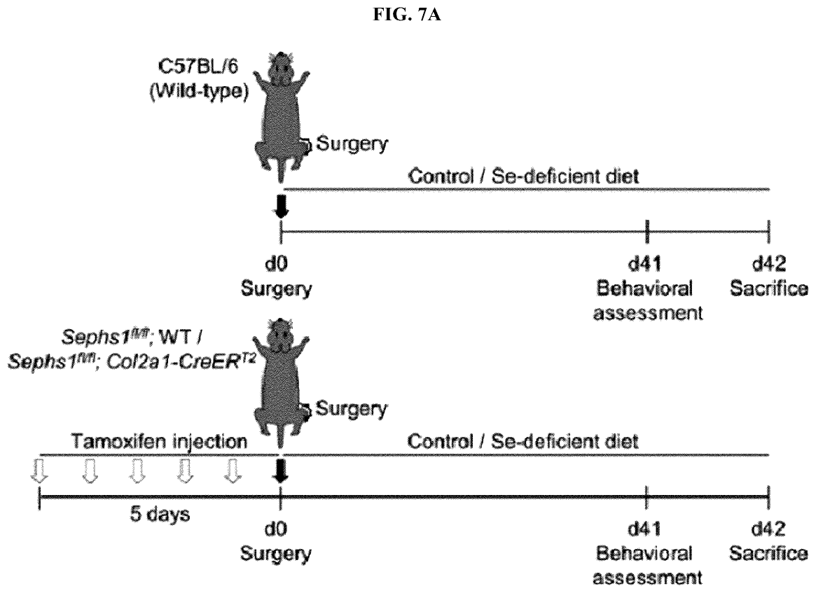

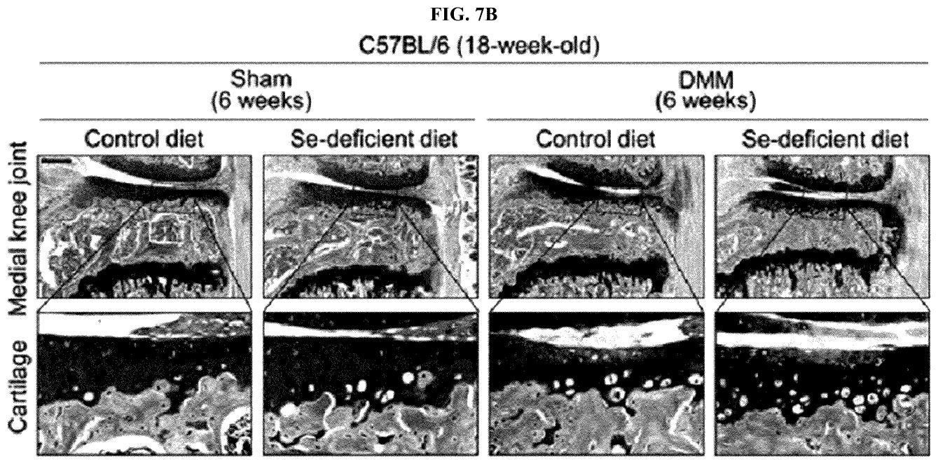

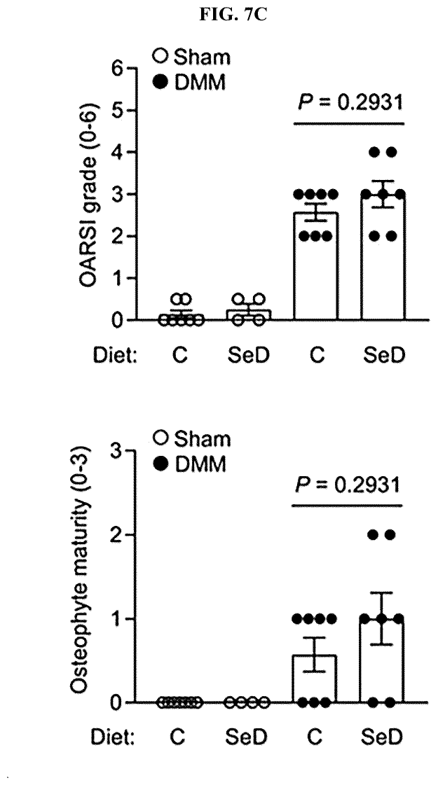

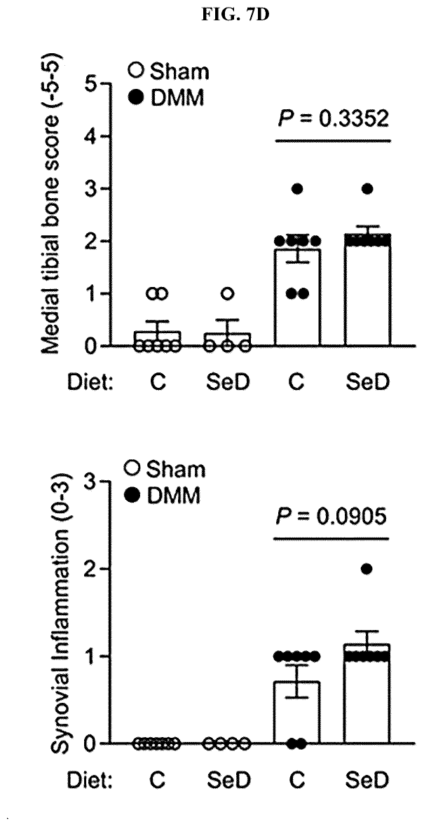

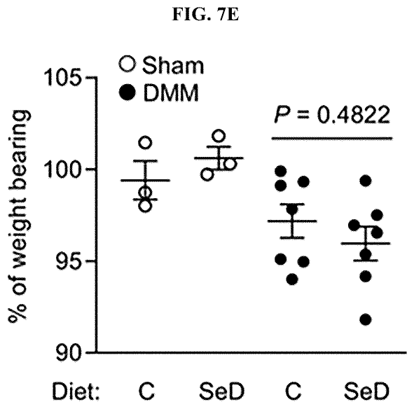

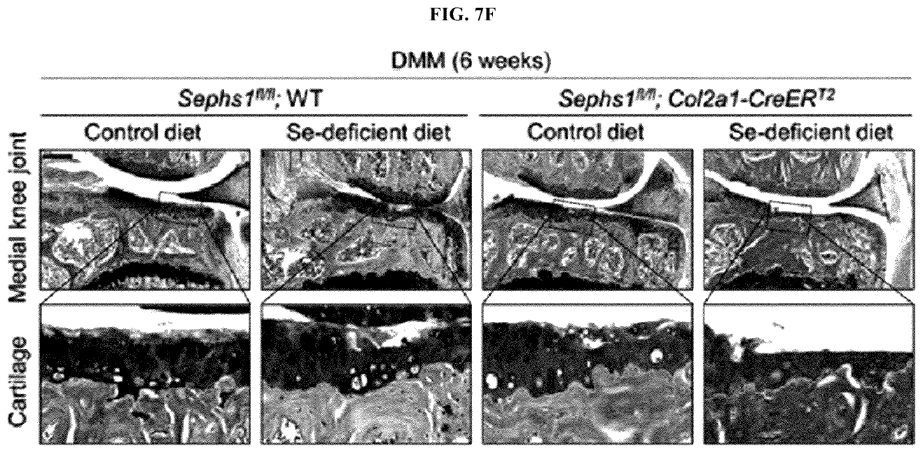

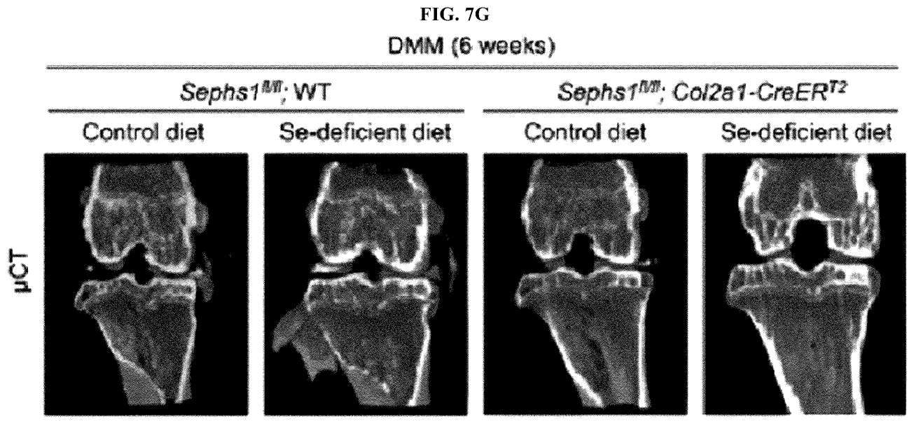

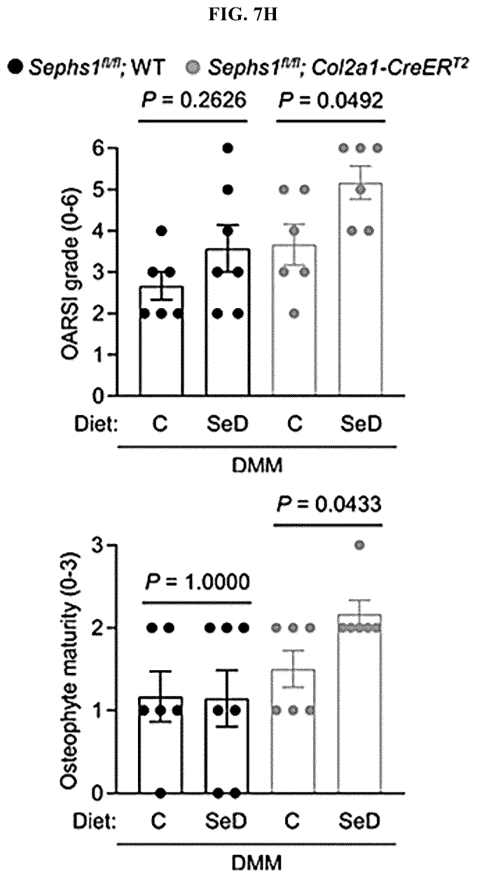

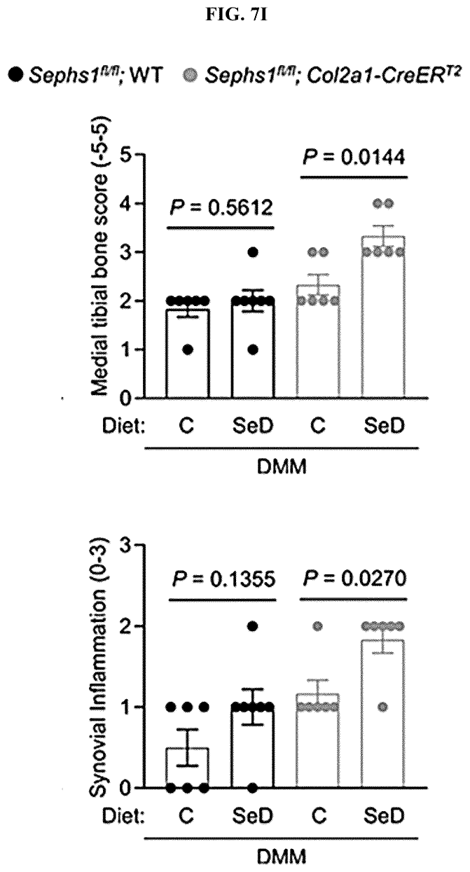

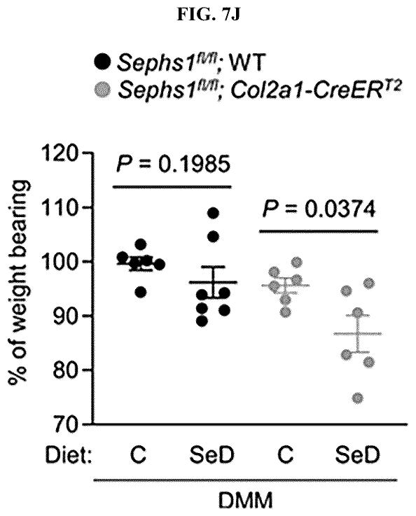

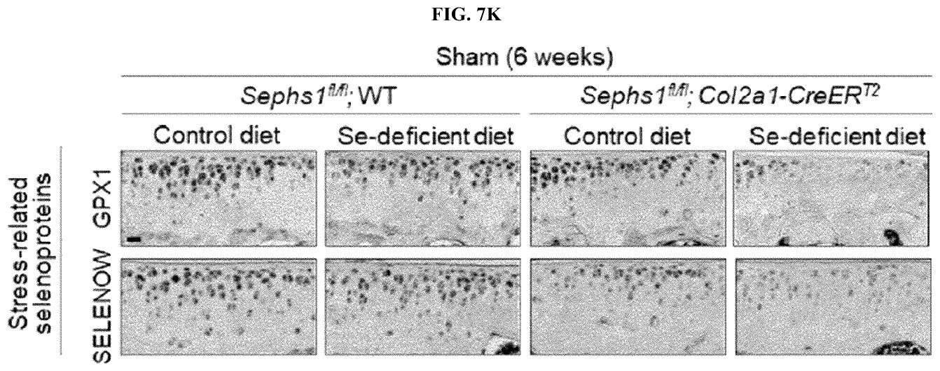

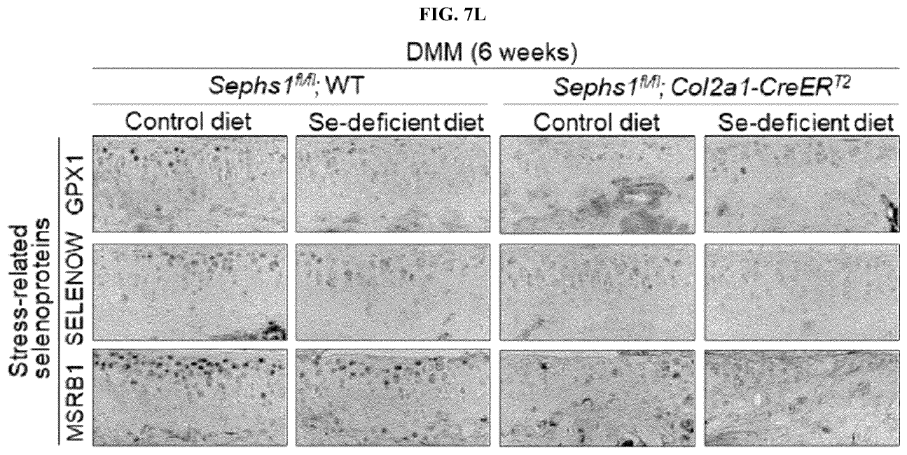

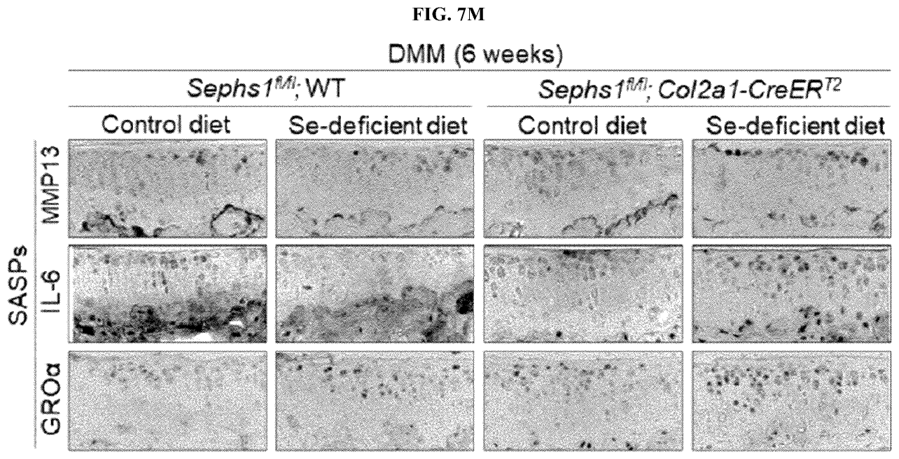

As in Experimental Example 3 above, mice were supplied with selenium-deficient feed, and phenotypes were analyzed when post-traumatic osteoarthritis was induced. As a result, it was confirmed that there was no significant difference in the osteoarthritis phenotype following the induction of osteoarthritis by the selenium deficiency itself, and no significant behavioral difference was observed. However, when dietary selenium was deficient together with SEPHS1 knockout, cartilage degeneration, subchondral bone sclerosis and synovial inflammation were significantly worsened 6 weeks after DMM, thereby confirming that the progression of osteoarthritis was accelerated. Consistent with these results, it was confirmed that the expression of stress-related selenoproteins and the expression of SASPs factors were further increased when SEPHS1 knockout and selenium deficiency were simultaneously treated. Further, behaviorally consistent results were obtained (FIGS. 7A to 7M).

### Example 6. Osteoarthritis Phenotype Analysis of Mice According to Selenium and Antioxidant Supply

Mouse chondrocytes were cultured according to Experimental Example 5, and after 2 days, selenium preparations or antioxidants were administered individually or in combination with H₂O₂. H₂O₂ was treated to reach 0 μM or 25 μM, sodium selenite used as a selenium preparation was treated to reach 500 nM, sodium selenate was treated to reach 500 nM, and selenomethionine was treated to reach 500 nM, respectively. As antioxidant preparations, N-acetylcysteine (NAC) was treated at 100 μM, ascorbic acid (aa) at 25 μM, and glutathione reduced (gsh) at 1 mM concentration. When the selenium preparation and the antioxidant preparation were mixed, these two preparations at the same concentration as those used alone were mixed and treated. After 72 hours of administration of the selenium preparation or the antioxidant preparation individually or in combination along with a defined concentration of H₂O₂, RT-qPCR was conducted according to the conventional method so as to analyze the expression of osteoarthritis-related factors.

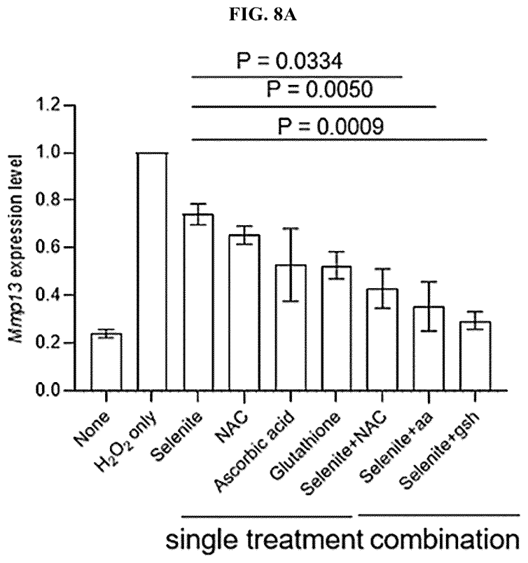

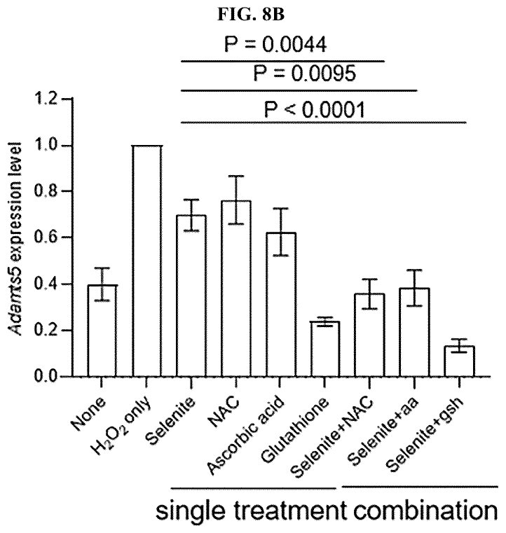

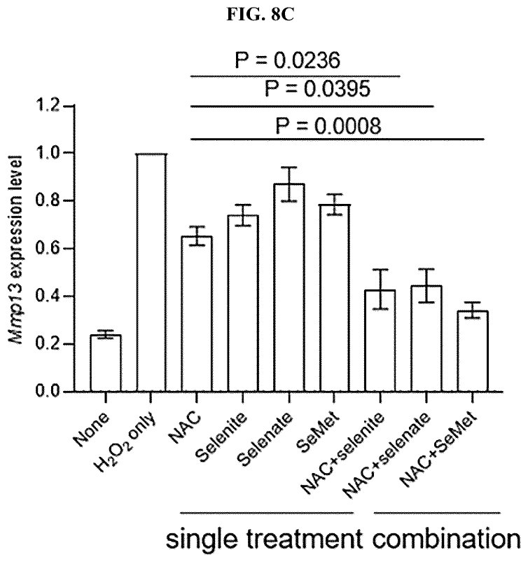

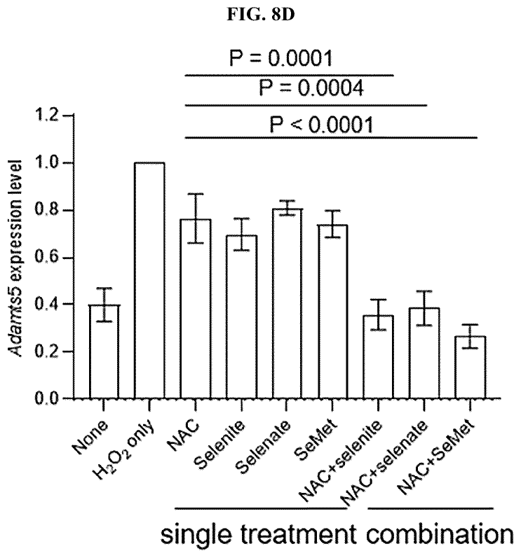

As a result, when only H₂O₂ was treated, it was confirmed that the increased expression of MMP13 or ADAMTS5, which is an osteoarthritis-related factor, was reduced by treatment with individual selenium preparations or antioxidants. In particular, when the selenium preparation and the antioxidant were administered together, it was confirmed that the expression of MMP13 or ADAMTSS was decreased similarly to the normal level, and it was confirmed that effects of suppressing the osteoarthritis phenotype were maximized during the combined administration (FIGS. 8A to 8D).

1. A composition comprising a selenium-containing material and an antioxidant.

2. The pharmaceutical composition according to claim 1, wherein the selenium-containing material is at least one material selected from the group consisting of selenite, selenate, selenomethionine (SeMet), and a salt thereof.

3. The pharmaceutical composition according to claim 1, wherein the antioxidant is at least one selected from the group consisting of N-acetylcysteine, ascorbic acid and glutathione.

4. The pharmaceutical composition according to claim 1, wherein the selenium-containing material is included in an amount of 0.1 nM to 10 μM in the composition.

5. The pharmaceutical composition according to claim 1, wherein the antioxidant is included in an amount of 1 μM to 10 mM in the composition.

6. (canceled)

7. (canceled)

8. (canceled)

9. (canceled)

10. (canceled)

11. A method for treating osteoarthritis, the method comprising:
administering a composition comprising a selenium-containing material and an antioxidant to a subject in need thereof.

12. The method of claim 1, wherein the selenium-containing material is at least one material selected from the group consisting of selenite, selenate, selenomethionine (SeMet), and a salt thereof.

13. The method of claim 1, wherein the antioxidant is at least one selected from the group consisting of N-acetylcysteine, ascorbic acid and glutathione.

14. The method of claim 1, wherein the selenium-containing material is included in an amount of 0.1 nM to 10 μM in the composition.

15. The method of claim 1, wherein the antioxidant is included in an amount of 1 μM to 10 mM in the composition.
